(19) United States
(12) Patent Application Publication
(10) Pub. No.: US 2012/0220486 A1
(43) Pub. Date: Aug. 30, 2012

(54) METHODS AND APPARATUS FOR BINDING ASSAYS

(75) Inventors: Javier Farinas, Los Altos, CA (US); Andrea Chow, Los Altos, CA (US); John Wallace Parce, Palo Alto, CA (US)

(73) Assignee: CAERUS MOLECULAR DIAGNOSTICS INCORPORATED, Mountain View, CA (US)

(21) Appl. No.: 13/505,455

(22) PCT Filed: Oct. 31, 2010
(86) PCT No.: PCT/US10/54904

§ 371 Date: May 1, 2012

Related U.S. Application Data
(60) Provisional application No. 61/257,013, filed on Nov. 1, 2009.

(30) Foreign Application Priority Data
Sep. 7, 2010 (US) 12877103

(51) Int. Cl. G01N 33/53; C40B 30/04

(52) U.S. Cl. 506/9; 436/501

(57) ABSTRACT

The present teachings relate to methods, systems, and apparatus for low cost label-free assay detection. The present teachings, in a variety of embodiments, employ opposing forces to detect signals which depend on the number of charges on and/or the size of a particle. The particle, which can be subjected to opposing forces, can have specific capture probes at its surface. As analytes of interest are captured by the particle, the number of charges on the particle surface and/or the size of the particle is changed. A particle parameter or kinematic property such as the position, velocity, acceleration or force of/on the particle can be measured, and results obtained relating, for example, to the present, absence, quantity, and such, of one or more analytes of interest. Various embodiments are described for efficient, high throughput assays of samples potentially including one or more analytes of interest, such as bioanalytes. As well, various embodiments are described wherein binding assays can be carried out without the need or use of extrinsic labels. A number of embodiments provide, for example, methods, systems, and apparatus for detecting analytes (such as nucleic acids, proteins, cells and other entities, particulates, and the like) in one or more samples. Also described are: detection of a single copy of a target biomolecule, such as DNA, captured onto a trapped (e.g., tethered) bead; protocols for fabricating encoded bead arrays for multiplex assays; and methods, systems and apparatus for efficient and specific capture of pathogen biomolecular markers onto bead-bound capture probes, as well as detection and measurement of such capture events.

### CROSS-REFERENCE TO RELATED APPLICATIONS

This application claims a priority benefit under 35 U.S.C. §119(e) from U.S. Patent Application No. 61/257,013, filed Nov. 1, 2009, which is incorporated herein by reference in its entirety.

This application is a continuation-in-part of U.S. patent application Ser. No. 12/877,103, filed Sep. 7, 2010, which is incorporated herein by reference in its entirety.

### FIELD

The present teachings relate to the field of biochemical, chemical, and cell-based assays. In particular, the present teachings relate to methods and apparatus for detecting analytes, such as nucleic acids, proteins, cells and other entities and particulates, in samples.

### BACKGROUND

Analysis of specific biological and non-biological materials, especially in low concentrations, is important in many fields, such as medical research, diagnostics, industrial processing, environmental monitoring, process and quality control, and forensics. For example, detection of specific nucleic acids or proteins in a patient sample is used to diagnose disease conditions, detect infectious organisms, and discover disease-causing genes.

In the United States, it has been reported that three of the ten leading causes of death in 2006 were infectious diseases; especially, chronic lower respiratory diseases, pneumonia/influenza, and septicemia. Infectious diseases accounted for more than 200,000 deaths. Of approximately 57 million deaths worldwide in 2002, over 25% were caused by infectious diseases. [See, respectively, M. Heron, “Deaths: Leading Causes for 2006,” National Vital Statistics Reports, 58:14 (2010); and, World Health Organization (WHO), “The world health report 2004—Changing history,” http://www.sho.int/shr/2004/en; each incorporated herein by reference].

Societal costs of infectious diseases are staggering. It has been reported that in 2000, treatment of non-AIDS STDs alone cost 6.5 billion dollars in the US annually [H. W. Chesson, J. M. Blandford, T. L. Gift, G. Tao, and K. L. Irwin, “The estimated direct medical cost of sexually transmitted diseases among American youth, 2000,” Perspect. Sex Reprod. Health, 41(4):217 (2009)]. Other reported yearly costs due to certain infectious diseases include (a) greater than 17 billion US dollars for community-acquired pneumonia, (b) 28 billion US dollars for influenza including direct medical expenses and lost potential earnings, (c) 2.4 billion US dollars for salmonella, and (d) 1.3 billion US dollars for Hepatitis B. [See, respectively, T. M. File Jr. and T. J. Marrie, “Burden of community-acquired pneumonia in North American adults,” Postgrad. Med., 122(2):130-41 (2010); C. Li and M. Freedman, “Seasonal influenza: an overview,” J. Sch. Nurs., 25(S1):4S-12S (2009); T. R. Callaway, T. S. Edrington, R. C. Anderson, J. A. Byrd, and D. J. Nisbet, “Gastrointestinal microbial ecology and the safety of our food supply as related to Salmonella,” J. Anim. Sci., 86(14S):E163-72 (2008); and, W. R. Kim, “Epidemiology of hepatitis B in the United States,” Hepatology, 49(5S):S28-34 (2009); each incorporated herein by reference].

Some of the known approaches for detecting low concentrations of analyte with good sensitively include the use of radioactive or fluorescent labels. Although these labeling methods have been used extensively, there are a number of drawbacks in practice. In the case of radioactivity, experimentation requires trained personnel, and reagent and waste disposal is expensive. In the case of fluorescence, experimentation requires relatively expensive reagents and detection instruments. Molecular diagnostic (MDx) systems using target analyte amplification (e.g., polymerase chain reaction (PCR)) tend to be sensitive and specific, but they are generally quite expensive, slow in getting to results, and difficult to use. A number of the existing diagnostics platforms based on lateral-flow immunoassays are sufficiently inexpensive, not overly burdensome to use, and typically provide results in an adequate timeframe, however, they generally suffer from low sensitivity and specificity. [See C. C. Ginocchio, F. Zhang, R. Manji, S. Arora, M. Bornfreund, L. Falk, M. Lotlikar, M. Kowerska, G. Becker, D. Korologos, M. de Geronimo, and J. M. Crawford, “Evaluation of multiple test methods for the detection of the novel 2009 influenza A (H1N1) during the New York City outbreak,” J. Clin. Virol. 45(3):191-5 (2009); and, M. A. Pfaller, “Molecular approaches to diagnosing and managing infectious diseases: practicality and costs,” Emerg. Infect. Dis., 7:312-318 (2001); each incorporated herein by reference].

There is an unmet need for a diagnostic system having acceptable sensitivity, low-cost, and capable to perform multiplexed pathogen detection, to assist in administering correct treatment in a timely manner. Such a system can be expected to significantly enhance the effectiveness of patient care in infectious diseases by enhancing the timeliness and accuracy of selecting appropriate treatment and lowering the overall costs of healthcare. [See, e.g., E. J. Baron, “Implications of new technology for infectious diseases practice,” Clin. Inf. Dis., 43:1318-23 (2006); J. Weile and C. Knabbe, “Current applications and future trends of molecular diagnostics in clinical bacteriology,” Anal. Bioanal. Chem., 394:731-42 (2009); and, B. H. Robertson and J. K. A. Nicolson, “New microbiology tools for public health and their implications,” Annu. Rev. Public Health, 26:281-302 (2005); each incorporated herein by reference].

### SUMMARY OF VARIOUS EMBODIMENTS

An exemplary and non-limiting summary of various embodiments is set forth next. The following various embodiments and examples are offered for illustrative purposes only and are not intended to limit the scope of the present teachings in any way.

In various aspects of the present teachings, opposed forces (also referred to as opposing forces) can be embodied in a variety of methods, systems, apparatus, and the like, for biochemical, chemical, and cell-based assays. Generally, according to various aspects of the teachings herein, two or more opposing forces can act upon at least one solid support (e.g., a particle, bead, and the like) associated with at least one recognition element of an analyte of interest. A kinematic property of the particle can be detected, and a first measurement can be obtained. Conditions can then be provided for an event to occur which, in circumstances where the analyte is present in the sample, can be effective to change the balance of forces. A second detection can be carried out, and a second measurement can be obtained. Analysis of the measurements can reveal whether or not there has been a change to the kinematic property. A change, or the lack of any meaningful change, can provide information about a sample potentially containing an analyte of interest, such as the presence, absence, quantity, etc., of the analyte.

Various aspects of the present teachings provide, for example, methods and apparatus for detecting one or more analytes, such as bioanalytes. In a number of embodiments, methods are provided including the steps of: (a) generating a set of particles which are associated with molecular or biomolecular recognition elements, such as capture probes capable of selective and specific binding with the one or more analytes; (b) applying an a first force, such as an electric force, and at least one opposing force to the particles in an aqueous medium; (c) for each particle, measuring a parameter or kinematic property which is a function of the charge on the particle; (d) contacting the probe-bearing particles with the sample, wherein specific binding of the target analytes to the probes takes place in the event the sample contains such target analytes; and (c) analyzing the resulting changes in the parameter to determine the quantity of analyte or analytes captured. A number of embodiments contemplate, for example, application of the opposing forces in an impulse, periodic, steady manner, or any combination thereof.

In some embodiments, for example, the particles can be beads, liposomes, micelles, lipid coated beads, polymer coated beads, oil droplets, detergent or lipid or polymer coated oil droplets, polymers, cells, and/or quantum dots. In certain embodiments, the analyte or analytes can include one or more nucleic acids, proteins, metabolites, cells and/or viruses. In a variety of embodiments, the opposing forces can comprise, for example, one or more optical, magnetic, entropic, hydrodynamic, gravitational, centrifugal or mechanical force, or any combination thereof. In some embodiments, at least one of the opposing forces can be generated, at least in part, by an optical trap, a magnetic trap, a cantilever, a centrifuge, a surface attached molecule, or any combination thereof.

According to a variety of embodiments, the parameter or kinematic property which is measured can include, for example, one or more of the position, velocity or acceleration of a particle. In certain embodiments, the parameter can be measured optically. In some embodiments, the position of the particles can be measured with fast quadrant diode detectors. In various embodiments, the position of the particles can be measured with an imaging detector (e.g., a fast CCD or CMOS camera, or similar array detector).

According to a variety of embodiments, the capture probe can comprise a nucleic acid, antibody, antigen, or any combination thereof. In some embodiments, the capture probe can comprise a synthetic recognition molecule. According to various embodiments, the number of particles measured can be 1, more than 1, more than 100, more than 10,000, more than 1,000,000. In a variety of embodiments, the number of capture probes on the particle can be 1, more than 1, more than 100, more than 10,000, more than 1,000,000. Various embodiments contemplate, for example, that the charge of the particles without the bound capture probes is close to (or made close to) neutral. In some embodiments, the change in charge and the measurement are performed concurrently. In certain embodiments, multiple bead types with different capture probes are used for multiplex analyte detection.

Further aspects of the present teachings relate to, for example, systems, apparatus, methods, and the like, for detecting one or more analytes, including the steps of (a) generating a set of beads which each have 1 to 100,000 copies of capture probes; (b) applying an AC electric field and an opposing force from an optical trap to the particles in an aqueous medium; (c) measuring the displacement of the bead from the center of the trap; (d) exposing the beads to a sample containing the analyte of interest; and (e) analyzing the resulting changes in bead position or Zeta potential to define the quantity of captured analyte.

A variety of embodiments of the present teachings provide systems, apparatus, methods, and the like, for detecting an analyte, including the steps of: (a) generating a set of beads which each have from about 1 to about 100,000, and in some embodiments from about 1 to about 1,000,000 (or more) copies of capture probes; (b) applying an AC electric field and an opposing force from a magnetic trap to the particles in an aqueous medium; (c) measuring the displacement of each bead from the center of the trap; (d) exposing the beads to a sample containing the analyte of interest; and (e) analyzing the resulting changes in bead position or Zeta potential to define the quantity of captured analyte.

Additional aspects of the present teachings provide systems, apparatus, methods, and the like, for detecting an analyte. In various embodiments, methods of the present teachings can include, for example, the steps of: (a) generating a set of beads which each have from about 1 to about 100,000 (or more) copies of capture probes; (b) applying an electric field (e.g., AC) and an opposing force by attaching the beads to a surface by a polymer in an aqueous medium; (c) measuring a kinematic property, such as the displacement of the beads from the central position; (d) exposing the beads to a sample containing the analyte of interest; and, (e) analyzing the resulting changes in bead position or Zeta potential to define the quantity of captured analyte.

Additional aspects of the present teachings relate to systems, apparatus, methods, and the like, for measuring the isoelectric point of one or more proteins. In a variety of embodiments, for example, methods according to the present teachings can include the steps of: (a) attaching proteins to a set of particles; (b) applying an electric force and opposing forces to the particles in an aqueous medium; (c) measuring a parameter or kinematic property which is a function of the charge on the particle; (d) changing the pH of the solution; and (e) analyzing the resulting changes in the parameter to determine the isoelectric point.

According to further aspects of the present teachings, a variety of embodiments relate to systems, apparatus, methods, and the like, for measuring enzymatic activity. For example, in various embodiments, methods of the present teachings can include the steps of: (a) generating a set of particles which are associated with capture probes; (b) applying an electric force and opposing forces to the particles in an aqueous medium; (c) measuring a parameter or kinematic property which is a function of the charge on the particle; (d) binding a mixture of product and substrate resulting from an enzymatic reaction to the probes; and (e) analyzing the parameter to determine the ratio of product and substrate in a sample. In some embodiments, for example, the product substrate mix can be obtained from a kinase reaction.

The present teachings provide, among other things, label-free methods for detecting nucleic acids, proteins, and biological and non-biological particles in samples using bead-based capture probes by subjecting the beads to opposing forces in order to measure the captured analytes. Properties of the analytes can also be detected (e.g. the isoelectric point, or the post-translational modifications on a protein, peptide or other substrate). In various embodiments, the present teachings provide for simultaneous detection of multiple analytes without the use of an extrinsic label such as radioactivity or fluorescence. Various embodiments of the present teachings provide, for example, increased sensitivity as well as reduced cost and time required to perform biochemical, chemical, and cellular assays for many applications, including, for example, medical research, drug discovery, clinical diagnostics, environmental monitoring, forensics, agricultural research and development, process and quality control, and homeland security.

### BRIEF DESCRIPTION OF FIGURES

These and other embodiments of the disclosure will be discussed with reference to the following non-limiting and exemplary illustrations, in which like elements are numbered similarly, and where:

FIG. 1 is a schematic representation of a particle experiencing an electrical force and an opposing optical force from a tightly focused laser beam (optical trap), according to various embodiments of the present teachings.

FIG. 2 depicts, in schematic fashion, plural forces acting on a particle, according to various embodiments of the present teachings; wherein the plural forces include (i) an electrical force, and (ii) opposed forces comprised of (a) a hydrodynamic force and (b) a restoring “spring-like” force.

FIG. 3 depicts, in schematic fashion, a bead being acted upon by plural forces, like that shown in FIG. 2; and additionally a specific binding event between a particle-bound biomolecular recognition element (such as an antibody) and an analyte (such as an antigen), with the capture of the analyte onto the bead-bound biomolecular recognition element changing the balance of forces, leading to a measurable change in bead displacement; according to various embodiments of the present teachings.

FIG. 4 is a schematic representation of a system and method for opposed-forces binding assays, according to various embodiments of the present teachings, wherein a charge-label moiety specifically binds to an analyte specifically bound to a particle-bound capture probe, and where surface charge measurements are made prior to such binding of the charge-label moiety and again after the charge-label moiety is cleaved and washed away from the particle-bound capture probe; thereby enhancing the difference between the two measured values over what the difference would otherwise be without use of the charge-label moiety; thereby increasing the efficiency and effectiveness of the binding assay.

FIG. 5 schematically depicts a sample-to-answer workflow for assay device, in accordance with aspects of the present teachings, wherein a sample is introduced into the device and a series of steps are carried out, all within the device, with the device ultimately outputting an answer pertaining to the sample for the user.

FIG. 6 shows, in schematic fashion, a particle bearing plural DNA capture probes at its surface, as well as a photocleavable barcode, for use in a point-of-care (POC), sample-to-answer work flow; according to various embodiments of the present teachings.

FIG. 7 is a schematic illustration showing a bead tethered to a surface, and bearing DNA capture probes specific for an analyte of interest; according to various embodiments of the present teachings.

FIG. 8 comprises a series of three illustrations, in schematic form, which depict events in a binding assay, as contemplated by various embodiments of the present teachings, whereby a tethered particle, being acted upon by opposing forces, captures a target nucleic acid by way of a sequence-specific surface-bound capture probe thereon, leading to a first change in position of the particle, and next a branched DNA moiety (bDNA) is hybridized to a complementary region of the probe-bound analyte, leading to still a further displacement of the bead from its original position; thus illustrating that binding of target nucleic acid coupled with branched DNA hybridization can result in very large change in displacement of the particle by enhancing the change in charge and thus detection sensitivity and specificity.

FIG. 9 is a schematic representation of a flow cell, as contemplated by various embodiments, for use in connection with opposing forces binding assays, according to the present teachings.

### DESCRIPTION OF VARIOUS EMBODIMENTS

Introduction

The present teachings provide, among other things, methods, systems, apparatus, and the like, to assay at least one sample for one or more analytes of interest.

In various aspects and embodiments, the present teachings can provide for acceptably rapid and accurate identification of infectious agents in a variety of circumstances, e.g., such as in near-patient settings to guide antimicrobial therapy, to prevent nosocomial spread, and to minimize the cost of hospital stays. As will be appreciated, the present teachings can help satisfy some or all of the unmet needs for a diagnostic platform with desirable attributes of low-cost, rapid turnaround time, high sensitivity and specificity, and ease of use.

As will become apparent, various of the embodiments and examples described herein, as well as modifications thereof that will be appreciated by those skilled in the art, provide acceptable sensitivity and specificity, approaching or like that of PCR-based assays, without the onerous operational complexity of PCR, thereby mitigating the potential for cross contamination. Furthermore, various embodiments in accordance with the present teachings provide good scalability and flexibility, as well as a high degree of multiplexing (for example, in various embodiments, within a range of about 3-20 target analytes per test). Platforms embodying aspects of the teachings herein can be easy to use by personnel in clinic or hospital laboratories without the need for extensive training, and provide a sample-to-answer workflow without necessitating user intervention. The time-to-result, in a variety of embodiments, can be expected to be no longer than tens of minutes, thereby, e.g., assisting a physician in making appropriate treatment decisions during a patient's single visit. As well, instruments and disposables, consistent with the teachings herein, can be expected to have a reasonably low cost permitting extensive use in near-patient settings.

As further described herein, the present teachings, in various aspects and embodiments, contemplate the use of an analyte-detection arrangement which employs opposing forces (e.g., electrical, hydrodynamic, and optical forces) in measurements and quantification of surface charge changes of a support element, such as a bead or particle, bearing analyte-specific probes due to specific binding of analytes from an infectious agent. Among other things, comprised of relatively low cost and readily available components, can facilitate realization of a low-cost, high-performance bench top system.

In general, various aspects of the present teachings provide for analysis of one or more analytes of interest, such as one or more bioanalytes, by use of opposing forces, such as for example an electrical force and at least one other source. For example, in some embodiments, at least one particle, bead, or the like, can be trapped (e.g., by way of an optical trap and/or a polymer tether) for use in an analysis, such as at an analysis region of an assay device. One or more capture probes can be associated with the trapped particle. The presence, quantity, etc., of one or more analytes of interest can be ascertained or determined by observing for a change in charge as one or more analytes from a sample are brought into contact with the one or more probes of the particle. At least one force (e.g., an electrical force) which has a strong dependence on the charge of a complex defined by the particle and any associated moieties (e.g., capture probes), and hydrodynamic and restoring, spring-like forces which have weak or no dependence on the charge of the complex, can result in detectable motion of the particle, which is a function of the net charge of the complex. Simultaneous detection of a large quantity of particles can provide high-throughput multiplexed assays for one or a plurality of analytes. Such a plurality of analytes can comprise, for example, a panel (such as a standardized panel).

Binding assays employing opposing forces can be carried out, in various embodiments, without the use or need of labels (e.g., fluorescent or radioactive labels). Being label-free, opposed-force methods, systems and apparatus for binding assays can avoid costly reagents commonly used with other known systems. As well, with binding assays that are free of extrinsic optical labels, systems for detecting particle motion can be relatively simple, thereby avoiding the use or need of complex, expensive detection assemblies often found in other systems. For example, various embodiments herein employ comparatively simple dark-field optics.

Additionally, opposed-force binding assays, as taught herein and described in a variety of embodiments, can avoid the need for amplification (e.g., as by polymerase chain reaction (PCR)), thereby simplifying sample preparation as compared to the present-day, PCR-based approaches.

Further discussion, description of various embodiments, and illustrative examples are provided herein.

Definitions

In considering the embodiments below, as well as elsewhere herein, the following definitions should be taken into account. As well, consideration should be given to what those skilled in the art would understand, within the overall context of the present teachings.

The singular terms “a”, “an,” and “the” include plural referents unless the content clearly indicates otherwise. Thus, for example, reference to “a nucleic acid” includes two or more such nucleic acids (e.g., as in a mixture), and the like.

The terms “hybridize” and “hybridization” refer to the formation of complexes between nucleotide sequences that are sufficiently complementary to form complexes, e.g., via Watson-Crick base pairing.

The term “cantilever” refers to a beam supported only on one end. For example, such cantilevers can be made, e.g., from micropipettes, from microfabrication of Si or polymers or from protein structures such as actin filaments.

The term “optical trap” refers to the use of light to apply forces to particles. Forces can be applied so that the position of the particle tends to a location in an aqueous medium due to a restoring force. For example, traps can be made by tightly focusing a laser beam or by opposing fiber optics.

The term “magnetic trap” refers to the use of magnetic field gradients to apply forces to one or more supports, such as one or more particles. Forces can be applied so that the position of a particle tends to a location in an aqueous medium due to a restoring force.

The term “polymer” refers to a molecule (often large) comprised of repeating structural units, or monomers, connected by covalent bonds. Examples of polymers include, without limitation, DNA, RNA, proteins, oligosaccharides, polyethylene, polyethylene oxide, etc.

A “polymer replicating catalyst,” “polymerizing agent” or “polymerizing catalyst,” is an agent that can catalytically assemble monomers into a polymer in a template dependent fashion; that is, in a manner that uses the polymer molecule originally provided as a template for reproducing that molecule from at least one or more suitable monomers. Such agents include, but are not limited to, catalytic proteins, such as enzymes, including nucleotide polymerases, e.g., DNA polymerases, RNA polymerases, tRNA and ribosomes.

The term “entropic force” refers to a force whose properties are primarily determined not by the character of a particular underlying microscopic force (such as, for example, electromagnetism), but by the whole system's statistical tendency to increase its entropy. A non-limiting example of an entropic force is the elasticity of a freely-jointed polymer molecule.

The terms “signal”, “parameter,” “signal parameter,” “property,” and “kinematic property” refer to a property of a support, such as a particle, bead, micro-carrier, and the like, such as position, velocity, acceleration or net force on the particle.

Generally, deoxyribonucleic acid (DNA) comprises polymeric strands of nitrogenous bases. The bases typically include purines (adenine and guanine, abbreviated as A and G, respectively) and pyrimidines (cytosine and thymine, abbreviated as C and T, respectively). Typically (e.g., under natural conditions), the strands configure as a double helix, with bases to the center (like rungs on a ladder) and sugar-phosphate units along the sides of the helix (like the sides of a twisted ladder). The strands tend towards complementarity (i.e., A pairing with T, and C pairing with G).

It is well established that nucleic-acid polymers generally comprise a sequence of linked-together nucleotide units or monomers (e.g., deoxyribonucleotide (DNA), ribonucleotide (RNA), and/or modifications or analogs thereof), and include a phosphate backbone that confers a net negative charge on the molecule. Such nucleic-acid polymers are often referred to in the relevant arts as “oligonucleotides” or “polynucleotides.” As is well known, for example in the field of electrophoresis, nucleic acids are polyelectrolytes whose physical properties and chemical reactivity are affected by the pH and ionic strength of a solution. In addition to the ionizable phosphodiester internucleotide bonds, the bases can be ionized or protonated depending upon the pH. The pK values of the nucleoside and sugar-phosphate backbone components of deoxyribo- and ribonucleic acids are well characterized. The pKa of the phosphate group, i.e., the measure of how readily that group will give up a hydrogen cation proton, is near 1. Thus, under most ionic condition, including physiological pH, the backbone will contain a single negative charge for each nucleotide unit, or two negative charges for a Watson-Crick pair of nucleotides in a double strand. (Zwolak, Michael and Di Ventra, Massimiliano (2008), Colloquium: Physical approaches to DNA sequencing and detection. Reviews of Modern Physics, 80 (1). pp. 141-165. ISSN 0034-6861.) Under physiological conditions of pH, the phosphodiester bonds are ionized, whereas the bases are in a neutral form, and thus the nucleic acid has an overall net negative charge.

Because each nucleotide is ionized, the charge-to-mass ratio of two different nucleic acid molecules will very closely agree. (Recombinant DNA principles and methodologies, edited by James J. Greene, Venigalla B. Rao. Published 1998, Marcel Dekker, New York). Under influence of an electric force, negatively charged nucleic acid molecules tend towards a positive pole in an electrical field.

As a brief aside, it should be noted that the terms “oligonucleotides” and “polynucleotides,” among others (e.g., “nucleic acid” and “nucleic-acid molecule”) often encountered in the relevant arts, are sometimes used to convey features or structural information (albeit, typically in a generalized, high-level fashion). For example, “oligonucleotide” is sometimes intended and understood to indicate generally short sequences, and “polynucleotide” is sometimes intended and understood to indicate generally long sequences (in other words, “oligonucleotides” tend be short, relative to “polynucleotides”). Notwithstanding the foregoing, it should further be noted that on many occasions such terms are used by those in the art in an imprecise fashion; without thought or intent of conveying features or structural information. In this regard, it is not uncommon for such terms to be used interchangeably. From knowledge and experience, persons regularly working and/or skilled in the relevant arts will appreciate the varied usage of these terms.

From knowledge, experience, and consideration of the overall context in which the terms as discussed above are used, those skilled in the art will appreciate the meaning of such terms herein. For example, in various contexts, the terms “polynucleotide,” “oligonucleotide,” and “nucleic-acid molecule” will be understood to include polymeric forms of nucleotides of any length (i.e., number of sequential bases), either ribonucleotide or deoxyribonucleotide.

Unless clear otherwise, terms herein such as “polynucleotide, “nucleic acid,” “oligonucleotide,” and “nucleic-acid molecule,” refer to the primary structure of the relevant molecules. Thus, for example, such terms include triple-, double-, and single-stranded RNA, as well as triple-, double-, and single-stranded DNA. As well, they include modified forms, such as by methylation and/or by capping, and unmodified forms. Generally, there is not intended any distinction in length between the terms “polynucleotide,” “oligonucleotide,” “nucleic acid,” and “nucleic acid molecule,” and these terms may be used interchangeably herein. These terms can include, for example, double- and single-stranded DNA, as well as double- and single-stranded RNA, DNA:RNA hybrids, and also various modifications, for example, labels (as known in the art), methylation, “caps,” substitution of one or more of the naturally occurring nucleotides with an analog; and modified nucleic acids, such as locked nucleic acids; as well as unmodified forms of polynucleotide or oligonucleotide.

The terms “protein”, “proteins”, “peptide”, “polypeptide”, and “lipoprotein” are used herein to include a polymeric form of peptide of any length. This term refers only to the primary structure of these molecules. Thus, the term includes natural or denatured conformations. It also includes modifications of these molecules, such as by fatty acids and/or by carbohydrates and/or by capping with a reactive group at the end for attaching it to another entity, and unmodified forms of the molecules.

As used in connection with an analysis of polymeric molecules associated with a support, plural forces are considered herein to be “opposing,” or “opposed to,” each other when at least two of the plural forces acting on the support include vector components that are pointing in opposite directions. As well, for purposes herein, at least one force of the plural forces is typically characterized by a dependence on (or, in other words, is a function of) the charge, and changes made to the charge, of the charged polymeric molecule(s) analyzed. In a variety of embodiments, such dependence is a strong one, and at least one other force of the plural forces is characterized by only a weak or no dependence on the charge of the charged polymeric molecule(s) analyzed. Still further, in various embodiments, the latter force is additionally characterized by a restoring, spring-like quality. The forces can include, but are not limited to, body and surface forces acting on the monomer units of the polymeric molecule(s) analyzed, and/or on the support. Exemplary forces comprise, without limitation, electrical, dielectrophoretic, mechanical, hydrodynamic, entropic, magnetic, optical, etc. The foregoing is provided for illustrative purposes. It is noted that, among other things, the choice of forces, their directions of application, and the magnitude of their strength can vary.

### Various Embodiments

Reference will now be made to a variety of non-limiting embodiments, various examples of which are illustrated in the accompanying drawings.

Aspects of the present teachings relate to systems, apparatus, methods, and the like, for assays using opposed-forces, comprising application of a force that depends on the charge of a complex for binding an analyte of interest, and further wherein such charge can be changed under appropriate conditions by a suitable event or means.

At the outset, it should be noted that “charge” is sometimes used as an indicator of (i.e., in a sense, as a proxy for) the presence of a molecule of interest. In some circumstances, it can be more natural to consider the forces acting on a molecule due to the overall or net charge of the molecule. Thus, for example, with regard to an analysis of a biological molecule associated with a support element (e.g., a particle bearing one or more oligonucleotide probes, incubated with a fluidic sample potentially including an analyte of interest comprising a nucleic-acid polymer capable of binding via hybridization with such probes), at least one of the opposing forces applied typically depends directly on the overall charge of the molecule. For example, with regard to a charged nucleic-acid molecule, at least one of the applied forces is typically characterized by a dependence on such charge. The charge, in turn, is a function of the number of nucleic-acid monomer units comprising the nucleic-acid molecule.

As further described herein, various embodiments provide, for example, methods, systems, sub-systems, apparatus, components, processes, assays, reagents, and the like, for assaying one or more analytes of interest (e.g., biological molecules), including various biological polymers, such as one or more peptides, proteins, enzymes, antibodies, strands of nucleic acids (e.g., nucleic-acid molecules, polynucleotides, oligonucleotides, DNA, RNA, etc.).

According to various embodiments herein, in general, a biological polymer such as one or more probes comprising polymeric nucleic-acid molecules capable of binding an analyte of interest in a sample is maintained on or proximate to a support element or micro-carrier, such as a small particle, bead (sometimes referred to as a microbead), or the like. In various embodiments, the support element can be trapped, fixed or otherwise immobilized. In a variety of embodiments the support element is, or can be, mobile (moving, or adapted for movement). The support element and associated probe(s), which collectively define a complex, can be disposed such that at least two opposing forces, when active, can act upon the complex. At least one of the forces has a dependence on the charge of the complex. The complex assumes a given state consistent with the environment about it, as provided in the system (e.g., a generally fixed or otherwise an initial location, or a velocity, acceleration, displacement, etc.), which environment includes applied opposing forces as well as charges associated with the support element itself and charges influentially proximate it (for example, charges of or relating to probes bound to the support element). The complex is then subjected to an event capable, under circumstances wherein the analyte is present, of changing the balance of forces and so effecting a change in a parameter or kinematic property of the complex (e.g., the perturbation in the balance of forces, for example, can move the complex to a different location, induce a change the velocity, etc.). Detection and measurement for a change in one or more such properties of the analyte-bound support element can indicate the presence, absence, quantity, etc. of one or more analytes of interest.

As previously indicated, the present disclosure provides a variety of embodiments of methods, systems, apparatus, and such, for label-free detection of biological and non-biological entities in a sample at low cost. In some embodiments, for example, a set of particles including one or more copies of a surface-bound capture probe is subjected to applied opposing forces. The opposing forces, in some embodiments, include an electrical force which is dependent on the number of charges on the particle and, for example, one or more of the following forces: optical, magnetic, hydrodynamic, entropic, dielectrophoretic, mechanical, gravitational or centrifugal. The forces opposing the electrical force are non- or weakly-dependent on the charges on the particle and can be dependent on other physical properties of the particle such as the effective size or hydrodynamic radius. In response to the applied opposing forces, one or more particle parameters such as position, velocity, or acceleration of the particle change due to a change in the number of captured entities on the particle. At least one of the relevant particle parameters is measured before the change, and then concurrently with and/or after the change. The analyte of interest can be captured through a variety of interactions, including, e.g., nucleic acid hybridization, antibody-antigen binding, ionic forces, hydrophobic interactions, van de Waals forces, and binding via shape recognition. In cases where a captured analyte does not add sufficient charge or size to detect, the change in a relevant particle parameter or property can be optionally enhanced (in other words, amplified) by introducing a third, highly charged entity interacting with the captured species.

In certain embodiments of systems and methods for the detection of analytes (such as bioanalytes, e.g., proteins, nucleic acids, cells and/or viruses in one or more samples) using opposing forces, a relevant particle parameter is measured with an optical detection system, such as a system capable of measuring transmitted, absorbed, scattered, polarized, phase-shifted and/or emitted light. For example, microscopes for brightfield, darkfield, phase contrast, DIC and/or fluorescence microscopy can be used. In various embodiments, the optical detection is capable of measuring the particle parameter for a plurality of particles arrayed on a substrate. The array can comprise, for example, more than 1, more than 100, more than 1,000, more than 10,000, and/or more than 1,000,000 particles. Some embodiments herein contemplate an array comprised of sub-arrays (i.e., an array of arrays). Regarding detection, various light detectors can be employed, such as quadrant photodiodes, or in some cases imaging systems (e.g. charged coupled devices or CMOS detectors, and the like). The detection system can be operably coupled to a computer, e.g., via an analog to digital converter, for transmitting detected light data to the computer for analysis, storage, data manipulation, etc.

While there are advantages provided by embodiments that avoid the use of extrinsic labels, such as fluorescent labels, such labels can nevertheless be included in systems and methods consistent with the present teachings, if desired. Thus, for example, in configurations employing fluorescent materials, the detector can include a light source that produces light at an appropriate wavelength for activating the fluorescent material, as well as optics for directing the light source through an optically clear detection window for observing material in the sample cell. The light source can be any number of light sources that provide one or more appropriate wavelengths, including, e.g., lasers, laser diodes, and LEDs. Other light sources can be used in other detection systems. For example, broad band light sources can be employed in light scattering/transmissivity detection schemes, and the like.

The selection of particle or bead materials can vary. For example, in various embodiments, materials such as glass, quartz, polymer, ceramic, metallic, paramagnetic, and composite materials can be used. In accordance with some embodiments, the support element can comprise a polystyrene bead or a gold particle

Capture probes can be associated with support elements by any suitable means. In a variety of embodiments, at least one probe comprising an oligonucleotide is bound, linked, fixed, attached, or otherwise associated with a bead or particle. Some embodiments contemplate, for example, different, respective sets of capture probes, with each set being carried by a subset of beads of a plurality of beads. As previously mentioned, in various embodiments, one or more probes can be configured to function as capture probes, with each being specific to a selected analyte of interest. In this way, one or more analytes of interest, to which the probes are specific, can bind with the probes. In a variety of embodiments, plural capture probes are associated with one or more beads, with the probes comprising nucleic acids (e.g. DNA, RNA or PNA), proteins (e.g. antibodies or enzymes), small molecules (e.g. antigens or metabolites) or other suitable selected molecules. In various embodiments, for example, sequence specific oligonucleotides comprise capture probes. One or more copies of such moieties can be associated with each bead. The association of capture probes with each bead can be achieved in a variety of ways; for example, through physical adsorption, covalent bonding, non-covalent binding, or other suitable means. In further examples of various embodiments herein, antibodies, such as monoclonal antibodies, can comprise capture probes, with such moieties being bound to particles by any suitable means; e.g., via carbodiimide chemistry by reacting with groups containing amines.

The number of copies of capture probes associated with a bead or particle can vary. For example, various embodiments contemplate (per bead or particle) only a single copy, or plural copies (e.g., up to about 100 copies, up to about 1,000 copies, up to about 10,000 copies, and in certain embodiments more than 10,000 copies).

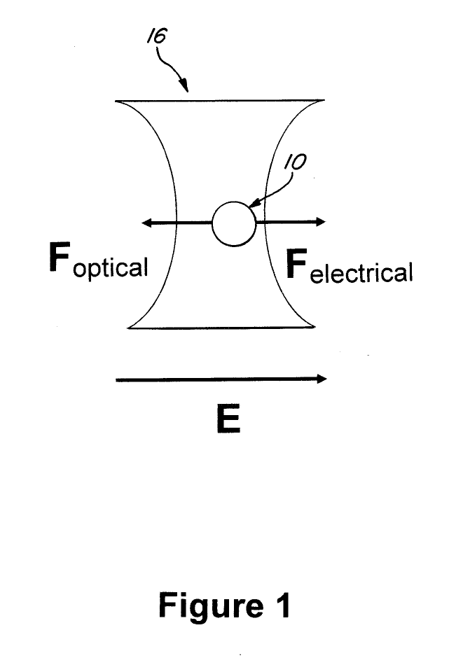

Further illustrating certain aspects of the present teachings, reference is now made to the embodiment depicted schematically in FIG. 1, wherein plural forces including an electrical force, F_{electrical}, and a restoring optical force, F_{optical}, are applied so as to act on a probe-bearing bead 10 and establish a signal parameter or kinematic property whose value depends on the number of charges on the bead and/or the size of the bead. In the illustrated arrangement, optical force F_{optical} comprises an optical trap 16. At his point, it is noted that an optical trap can be created, for example, by tightly focusing a laser beam or by opposing fiber optics. According to various embodiments herein, forces can be applied, for example, so that the position of the particle tends to located at, and stay within the region of, an analysis site in an aqueous medium due to at least one restoring force such as, among other ways, an optical trap.

With continued reference to FIG. 1, the forces F_{electrical} and F_{optical} generate or establish a kinematic property relating to particle 10 which is detected and measured, with the value being dependent on the number of charges associated with the particle. A fluidic sample containing, or potentially containing, an analyte of interest to which the probes of particle 10 are specific is contacted with the trapped particle. Re-measurement and analysis of the kinematic property which depends on the charge of the particle can be used to assay for an analyte of interest. The number of charges of particle 10 can be changed, for example, upon an event wherein the analyte of interest is in fact present in the sample, such that the probes and analyte specifically bind one another. In the event of the latter, the bound (captured) moieties change the charge.

It is contemplated herein, in a variety of embodiments, that the electrical force has a strong dependence on the charge of the bead complex. In various embodiments, a strong dependence on the number of charges is determined to exist when a plot of the force versus charge would show a high slope; whereas, a force with a weak dependence which would show a low slope. Thus, in a variety of embodiments, “strong” and “weak” are considered relative to one another in a given system. And, as stated elsewhere herein, in various embodiments the one or more forces, other than the force that exhibits a dependence, show only very little to no such dependence. In some embodiments, where a strong force is indicated by a plot of the force versus charge showing a high slope, such high slope is relative to a force with a weak dependence which shows, for example, about ⅓ or less, about 1/10 or less, about 1/100 or less, the slope for the strong dependence.

In various embodiments of the present teachings, at least two forces are used, including a first force and a second force, with the second force opposing the first force (such as shown and discussed in connection with the embodiment of FIG. 1). Forces oppose each other when the forces have a vector component in opposite directions. In some embodiments, one or more forces in addition to the second force also oppose the first force. In many embodiments, it can be advantageous to include an electrical force among the forces employed as a means of applying a force which depends on the charge of the complex. In various embodiments, including certain embodiments described next, at least three forces are employed, including (i) an electrical force, and (ii) two additional forces, with at least one of the latter forces being providing a restoring force (e.g., a “spring-like” restoring force).

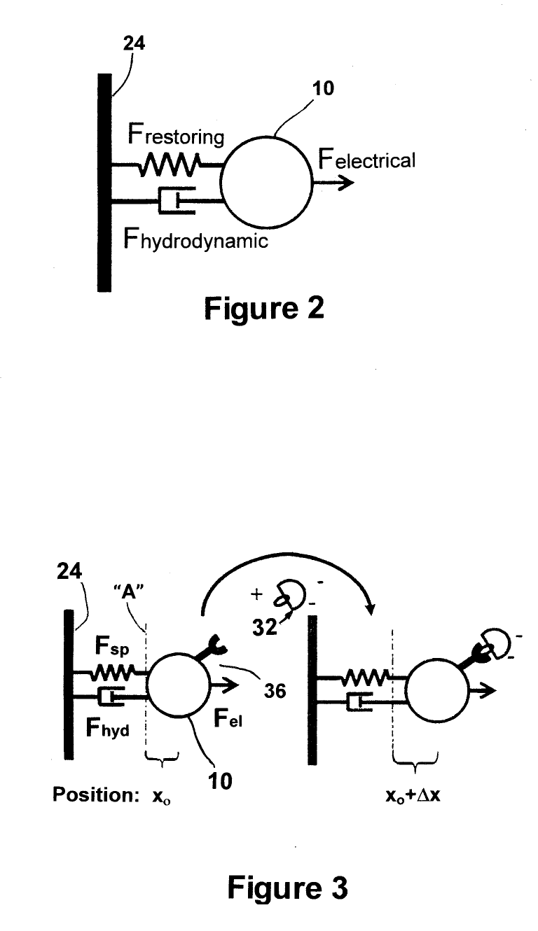

Referring to FIG. 2, a bead is schematically depicted at 10, and three forces are schematically shown which can act on the bead. FIG. 2, in particular, depicts three applied force vectors. As shown, the forces include (1) an electrical force, Fₑₗ, characterized by being directly dependent on the total (net) electrical charge the bead (or complex comprising the bead); and, additionally, (2) two opposing forces comprising (a) a hydrodynamic force, F_{hyd}, and (b) a spring-like restoring force, Fₛₚ.

In various embodiments, the means for a restoring force connects or links a bead or particle to an appropriate surface. In FIG. 3, for example, a means for a restoring force (Fₛₚ) is attached to a suitable surface, such as 24, by any suitable means. In some embodiments of the present teachings, a polymeric molecule, such as a biological polymer, is employed as a tether providing a restoring force. Various embodiments, for example, contemplate employing an oligonucleotide as a tether providing a restoring force; such as double-stranded DNA (dsDNA). Various embodiments contemplate other biological polymers, as well. For example, a tether can comprise a protein providing a restoring force. Further embodiments contemplate that a tether can be comprised of one or more non-biological polymers. For example, among other things, a tether can comprise polyethylene oxide.

In a variety of embodiments, such as shown schematically in FIG. 3, an antigen 32 can be captured by an antibody, such as monoclonal antibody (mAb) 36, attached to a bead 10, resulting in a change in the position of the bead under the applied opposing forces. Quantification of the displacement of the bead can be used to determine the total amount of antigen captured which is proportional to its concentration in a prepared sample. For example, in an arrangement comprising a plurality of beads, with each bead influence by a trapping force (e.g., by way of an optical trap, or a polymeric tether) at a unique respective site in an analysis region, and with an electric force acting upon the plurality of beads, the positions of the beads can be used to measure the Zeta potential of the beads. This can be used to measure the change in the charge on beads involved in binding events, wherein a charged moiety becomes associated with a bead. In various embodiments, such a system can be used, for example, to detect the incorporation of one or more specific analytes of interest (e.g., DNA or RNA) via plural nucleic-acid probes bound to each of the beads, or another molecular entity (including but not limited to antibodies, antigens, proteins, polysaccharides, lipoproteins, mammalian cells, bacterial cells, virus particles, metabolites, organic contaminants, inorganic particulates, etc.) using a suitable biological or synthetic recognition and capture probe.

With reference again to FIG. 3, and, particularly, with attention to the sequence of events shown, from the left to the right, it can be seen that upon incubating a probe-bound bead 10 with a sample containing an analyte of interest, such antigen 32, the antigen and bead-bound antibody can specifically bind one another. A detection and measurement system, including, e.g., a quadrature photodetector, (not shown) can be employed to detect and measure a consequent change in an affected (i.e., by the binding event) kinematic property of the particle. Notably, in FIG. 3, beginning from the left-hand side of the figure, particle 10 is initially located at a position x₀, directly adjacent and tangential to the vertical dashed reference line, denoted as “A,” extending substantially parallel, and in spaced-apart relation to, surface 24. Upon occurrence of the binding event, the balance of forces changes, and particle 10 moves rightwards (when looking at the figure), until once again the various forces balance and it locates at a new position, x₀+ΔX (as can be seen towards the right-hand side of FIG. 3).

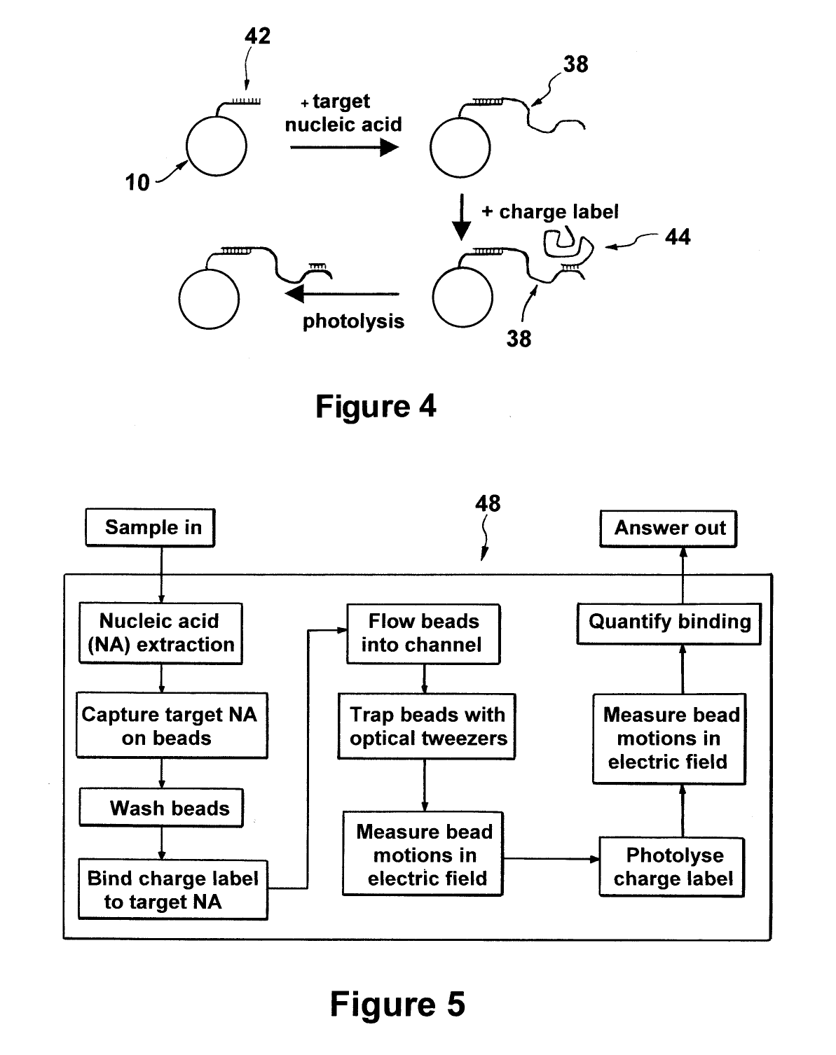

In place of an antibody-bound bead, an arrangement quite similar to that depicted in FIG. 3 can instead utilize oligonucleotide capture probes bound to beads, with each capture probe being adapted for specific hybridization with a target analyte of interest. A process, e.g., such as just described and as depicted in connection with FIG. 3, can be carried out employing such an arrangement. In this regard, for example, reference is now made to FIG. 4, which schematically depicts such a system and process. In FIG. 4, one or more copies of an analyte of interest, such as a target nucleic acid 38 derived from a pathogen, can be detected, for example, by (a) providing conditions suitable for specific binding (here, hybridization) between the target nucleic acid and an oligonucleotide probe 42 carried by bead 10, (b) optionally, binding a target-specific charge label 44 to the probe-bound target nucleic acid, (c) capturing individual beads in an optical trap (not shown in FIG. 4), and (d) while applying forces comprising both the optical trap and an electric field, detecting and measuring the bead surface charge at a first time, while then charge label is bound to the target analyte and at a second time, after the charge label has been cleaved from the bead-bound target nucleic acid. Various means can be employed to effect cleavage of the charge label, e.g., photo-triggered release using a photolabile charge label. It will be appreciated by those skilled in the art that the presence of the charge label, which is negatively charged, during the first measurement, combined with the absence of the charge label during the second measurement, can serve to enhance the magnitude of change to the kinematic property exhibited by the bead, thereby facilitating detection and measurement.

FIG. 5 provides a flow chart comprising various steps of an embodiment much like that shown and described in connection the embodiment of FIG. 4. In FIG. 5, it should be noted, the diagram schematically depicts the steps integrated inside a sealed disposable cartridge, 48, to yield a sample-to-answer workflow.

Various embodiments contemplated herein, which embodiments are generally like that, or similar to, the process flow depicted in FIG. 5, provide a point-of-care, sample-to-answer work flow, which can include the steps of: (a) extracting pathogen nucleic acids (RNA or DNA) from samples; incubating purified nucleic acids (NA's) with beads, such as bead 10 in FIG. 4, which are attached with DNA capture probes, as at 42; washing the beads and incubating with specific photocleavable signal amplifiers; flowing an aliquot of beads into a microchannel of an assay device and immobilizing individual beads with respective optical traps; measuring bead motions in response to applied E (X1); photolysing the charge labels, and re-measuring bead motions (X2); releasing beads from the optical traps; introducing another aliquot of beads and repeating opposing-force measurements to completion; then determining the amount (X1, X2) of target NA's captured based on ratiometric measurements of bead motions.

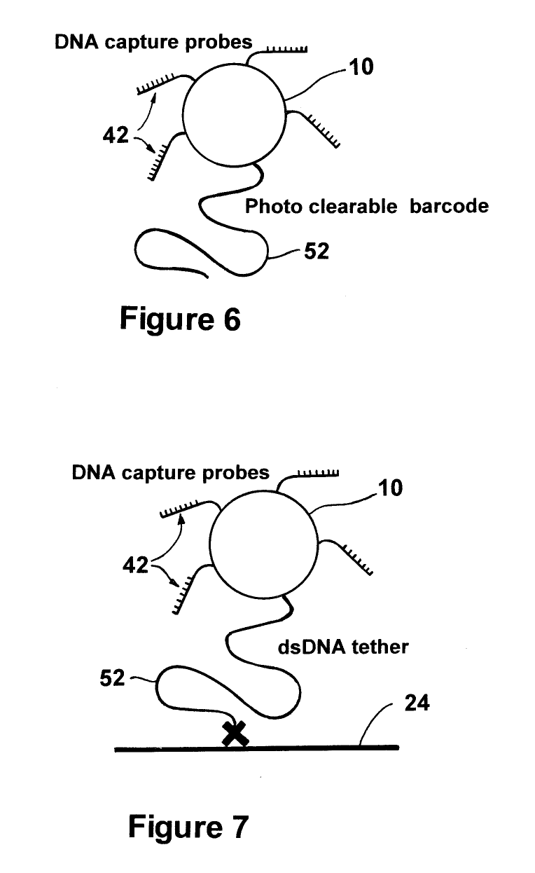

Alternative embodiments contemplated herein, which embodiments provide a point-of-care, sample-to-answer work flow for multiplexing analytes, which can include the steps of: (a) extracting pathogen nucleic acids (RNA or DNA) from samples; incubating purified nucleic acids (NA's) with beads, such as bead 10 in FIG. 6, which are attached with DNA capture probes, as at 42, and at least one photocleavable barcode, such as 52 in FIG. 6; washing the beads and incubating with specific signal amplifiers; flowing an aliquot of beads into a microchannel of an assay device and immobilizing individual beads with respective optical traps; measuring bead motions in response to applied E (X1); photolysing the barcodes, and re-measuring bead motions (X2); melting and releasing target NA's, and re-measuring bead motions (X3); releasing beads from the optical traps; introducing another aliquot of beads and repeating opposing-force measurements to completion; then determining the type (X1, X2) and amount (X2, X3) of target NA's captured based on ratiometric measurements of bead motions.

Approaches such as those described above can provide for a low-cost diagnostic point-of-care product concept using opposed-forces sensing, of the present teachings, to detect target nucleic acids in a multiplexed format using bead barcodes. Such approaches can be applied, among other ways, in connection with infectious disease detection, cancer mutation detection, and other diseases with markers suitable for binding interactions with capture probes. Bead traps can be employed, e.g., using optical, magnetic, or dielectrophoretic forces.

In various embodiments, bead barcoding is employed in combination with opposed-forces detection, according to the teachings herein, in a fashion providing for multiplex assays. Optical (fluorescent and non-fluorescent) and magnetic barcoding of microbeads have been useful for multiplexing assays up to hundreds and thousands of fold. When such technologies are combined with opposed-forces detection of the present teachings, with or without the use of a restoring force, similar enhancement in assay multiplexing can be performed readily as a specific barcode ID is correlated with a specific capture probe attached to the surface of the beads. As contemplated by various embodiments herein, sequence-specific DNA attached to the beads can also be used as a barcode. In this case, specific hybridization with known capture sequence after the assay is completed is used to identify the beads. Barcoding of beads can be used to avoid the use for spatial encoding for multiplexed assays, making the use of a restoring force to localize the beads optional in some embodiments, herein, employing opposed-forces detection.

As those skilled in the will appreciated, binding assays can sometimes be limited by background signals caused by non-specific binding of a signal generating moieties to surfaces. For example, in sandwich ELISA assays, fluorophore-labeled antibodies which non-specifically adsorb to surfaces (i.e. not by virtue of specific binding to an antigen/antibody complex) can yield a fluorescence signal which is essentially indistinguishable from the signal from specific binding. In the case of opposing forces sensing, one or more assay components other than the analyte of interest may in some circumstances bind nonspecifically to a binding complex and, in general, cause the number of charges on the complex to change. This could lead to a change in charge which is not dependent on the binding of the analyte of interest. While techniques exist to reduce non-specific binding to very low levels, background signals from non-specific binding may nevertheless limit assay performance.

One way to limit undesirable effects of nonspecific binding is to use a detection agent having a detection moiety coupled by a cleavable linkage to a detection label. In this case, a differential measurement is made in which the amount of label associated with an analyte complex is measured before and after cleavage of the label. This can essentially eliminate background signals that may otherwise be caused by non-specific binding of the label itself (since if the label is non-specifically adsorbed cleavage of the linkage does not result in a change in the amount of label associated with the analyte complex).

Embodiments employing an approach like or substantially similar to those just described can include a number of features which tend to make them well suited for near-patient diagnostic platforms. For example, the charge label can serve to amplify the detected/measured signal without requiring amplification of the nucleic acid itself. Thus, in various embodiments, single molecule sensitivity can be obtained without the degree of complexity, risk of contamination, need for complex fluorescence detection optics and relative slowness generally associated with amplification-based methods, such as PCR. The differential bead measurement before and after photocleavage of the charge label substantially minimizes non-specific binding effects, thus enhancing sensitivity and specificity. The bead binding and measurement operations can be combined, for example, on a microfluidic chip with a nucleic acid extraction module to provide an integrated system capable of sealed, sample-to-answer operation. The integrated sample preparation together with the lack of enzymatic reactions (particularly, e.g., for reverse transcription of RNA to DNA) can provide for the rapid generation of an analytical result. As well, the extracted nucleic acid can be incubated in multiple channels with beads bearing a variety of pathogen-specific capture probes to generate a multiplexed read-out.

A number of additional aspects, features, and variations which are contemplated for use in various embodiments consistent with the present teachings will now be discussed.

In a number of embodiments, at least two forces in addition to a first (electrical) force are employed; for example, the hydrodynamic and restoring forces as shown and described in connection with the embodiment of in FIG. 3. It is noted that such additional forces do not depend strongly on the number of charges associated with the assayed analyte(s). Rather, in such embodiments, they depend only weakly, or not at all, on the number charges associated with the analyte(s).

In various embodiments, tethered polymers that can act in a spring-like manner hold beads proximate to a surface, such as an analysis region or chamber of a cartridge or microfluidic device. In some embodiments, arrays comprised of a plurality of beads are formed by attaching the beads to a surface in an analysis region or chamber by way of biotinylated polyethylene glycol (PEG) linkers which bind to a surface patterned with streptavidin. The streptavidin can be printed by any suitable means, such as by soft lithography printing methods. In some embodiments, the beads of an array can be biased to one side with a DC component of an electric field used during detection, thereby stretching the respective tethers to yield spring constants of ˜0.1 pN/nm. Bead motion can be detected in a variety of ways. In various embodiments, for example, an imaging array detector (e.g. CCD or CMOS cameras) is employed. In some embodiments a quadrature photodetector assembly is utilized.

A restoring force, as contemplated herein, can be provided by any suitable means. For example, in a variety of embodiments, a means providing a restoring force comprises a tethering construct. Generally, in a wide variety of embodiments contemplated by the present teachings, a construct is selected that is, or can exhibit in pertinent circumstances, a “spring-like” nature or characteristic. In various embodiments, for example, a construct comprising a means for tethering is selected or adapted to be functional for linking a particle or bead to a suitable surface or other appropriate structure. A means for tethering means can be selected, according to various embodiments, for suitability to span a region separating a probe-bearing particle and a solid support, such as the surface, at 24, in FIG. 3. In a variety of embodiments, a means for tethering connects, or links, a probe-bearing particle with a solid support, such as a surface of a device, e.g., a surface or feature inside a flow channel, reaction chamber, and the like. In various embodiments, tethering means can be selected or adapted to be at least somewhat resiliently flexible. In various embodiments, a means for tethering is selected or adapted to be substantially stable under conditions for a binding event that effects a change in the number of charges associated with a particle to which the means for tethering is attached. Similarly, the means for tethering is selected or adapted to be substantially stable upon a change to one or more kinematic properties that accompany such change to the charge of an associated bead complex (e.g., such as brought about by a specific binding event, as described above). In some embodiments, tethering means are selected for characteristics such as low cost, and if desired, suitability for fabrication of large bead arrays for high-throughput assays and experiments. In some embodiments, with regard to the latter, polymer tethers can be employed.

It is contemplated herein that the choice of forces, their directions of application, and the magnitude of their strength can vary. In some embodiments, a time-varying force (e.g. an AC voltage) is applied to enable the use of Fourier techniques to facilitate precision in measuring the particle parameter. In certain embodiments, for example, a force can be turned on and off quickly and the response of the particle parameter to this impulse measured. In some embodiments, the forces are applied continuously or in a steady-state manner.

The forces can be, but are not limited to, body and surface forces acting on, e.g., complexes comprising probe-bound beads or particles. According to various embodiments, suitable forces can include, for example, magnetic forces (e.g. magnetic tweezers), electrical forces (e.g. electrophoretic, dielectrophoretic), optical forces, hydrodynamic forces, entropic, mechanical, gravitational or centrifugal forces. In some embodiments, an electrical force, which depends on the number of charges associated with the beads, is opposed by a hydrodynamic force on a bead and a force induced by stretching a polymer that tethers the bead to a surface. Assays can be accomplished, for example, by detecting for changes in the motion of the bead as the balance of the forces changes with binding events. In many embodiments, the configuration of opposing forces is selected to enhance detection sensitivity, accuracy, precision, and/or speed of measurement.

In some embodiments, the forces are applied in a way to generate a spatially separated array of particles; which, among other things, can facilitate multiplexing. Arrays comprising a plurality of particles include, for example, at least more than 1, and in various embodiments greater than about 100, greater than about 10,000, up to about 1,000,000 particles, or more. Forces such as optical, magnetic, dielectrophoretic, and the like, can be useful for creating arrays of particle traps, as well as tethers anchoring particles to a surface. In some embodiments, a trap employed for probe-bound particles herein comprises a dielectrophoretic trap [See, e.g., Lin, Yen-Heng, Chen-Min Chang, and Gwo-Bin Lee. 2009. Manipulation of single DNA molecules by using optically projected images. 17, no. 17: 15318-15329. http://www.ncbi.nlm.nih.gov/pubmed/19688010]. In various embodiments, an array of polymers is spotted onto a substrate (e.g., a surface of a flow cell or reaction chamber). In certain embodiments, for example, an array of polymers is spotted onto a substrate employing a polymeric spotting composition. Such composition can, for example, bind to the substrate and the particles and generate an entropic force to oppose an electrical force. In some embodiments, an opposing force can be provided by a gel which envelops one or more particles.

Further regarding means for tethering particles to a substrate, various embodiments herein contemplate, for any given particle, one tether comprising a suitable polymeric molecule. The tether can bind, for example, at (or near) one of its ends to the substrate, and at (or near) its other end to a respective particle, whereby an entropic force can be generated that can oppose, for example, an applied electrical force. In accordance with various embodiments, arrays of tethered particles can be formed that comprise, for example, more than 1, more than about 100, more than about 10,000, more than about 1,000,000, and/or more than about 10,000,000 particles.

It should be appreciated, at this point, that opposing-force binding assays, as taught herein, include a variety of embodiments that do not require or employ the use of optical labels (i.e., the binding assays can be label-free in that regard).

In various embodiments, a tether chain can anchor a bead to a surface of a flow cell, such that the bead can act as a probe for target analytes as a sample passes through the flow cell and contacts a probe-bearing tethered bead. Upon a binding event, a change takes place in the balance of opposing forces (e.g., electrical, hydrodynamic, and restoring forces), resulting in changes in bead displacement or velocity in opposed-forces detection of the present teachings. It is recognized that DNA is capable of providing a spring-like restoring force. The use of DNA herein as a polymeric facilitates customization of the length of the tether chain and, as well, provides for a wide selection of linker chemistry. Therefore, in a variety of embodiments DNA is employed as a tethering means, providing a number of desirable benefits. In a variety of embodiments, DNA strands are configured to anchor respective beads and to provide a restoring force in opposed-forces detection, according to the present teachings.

Further with regard to the use of DNA as a tether means, when a specific sequence of interest is incorporated in the tether DNA, it can also be used as a probe to detect target DNA and RNA of interest in sample, according to various embodiments. For example, an exemplary method to detect target capture, contemplated herein for use herein, is to measure changes in the stiffness of the chain using opposing forces when the bead charge and hydrodynamic drags remains constant while the elastic constant of the spring changes as a result of target capture (which can optionally be followed by a ligation step). In further embodiments, a tether comprising single-stranded DNA (ssDNA) can be configured to form one or more secondary structures (such as a hairpin structure by spontaneous hybridization of one part of the DNA to another), which structure(s) are disrupted upon, and by, capture of a target nucleic acid. The destruction of the secondary structure(s) can change the length of the tether and can be measured as changes in bead displacement under an electric field.

Next, embodiments of the present teachings are described which provide, among other things, microfabricated flow cell geometries to control the reproducibility of hydrodynamic interactions with the cell walls while measuring the electric field induced motions of tethered beads during analyses employing opposing forces, including binding assays and sequence analysis by use of opposing forces

In various embodiments of sequencing by use of opposing forces, taught in co-pending U.S. patent application Ser. No. 12/877,103, filed Sep. 7, 2010 (incorporated herein by reference in its entirety), an array of tethered beads covered with DNA templates can provide for highly parallel sequencing reactions, performed simultaneously, thus increasing analysis throughput. In opposed-force binding assays and detection of target analytes, of the present teachings, bead array yields multiplexing of target analytes. In both such sequencing and such binding assays, the tether provides spatial localization of beads as well as a restoring force to one or more additional forces, e.g., electrical and hydrodynamic forces, to increase measurement sensitivity. The tethered beads can be subjected to a DC electric field to stretch the tether and an AC electric field to probe charge density of beads. If the stretched tethers bring the beads too close to the substrate surface causing high sensitivity to hydrodynamic interactions with the flow cell walls, certain embodiments contemplate the use of streptavidin-labeled beads bound to the surface which can capture the tethered beads. This places a spacer between the substrate surface and the tethered beads to minimize hydrodynamic interactions between each bead and the surface. In some embodiments, the floor of the bead chamber can be microfabricated with post arrays with cross-sectional dimensions on the order of the bead diameter and an appropriate spacing on the order of the stretched tether. As the tethered beads are anchored on top of the posts, the stretched tether beads will not be subject to high hydrodynamic interactions with the top and bottom of the bead cells.

Turning now to support elements, such as particles and beads, a support element associated with at least one binding moiety (e.g., an oligonucleotide or antibody probe, among others) can be disposed such that a plurality of opposing forces can act upon it. The support element can be comprised of any of a variety of suitable materials and, as well, be configured in any suitable shape or form. Informed by the teachings herein, suitable support elements can be selected by those skilled in the art.

It is noted that, in connection with support elements, no particular distinction is intended between the terms “particle” or “bead,” or the like; unless otherwise clear. Such terms, herein, may sometimes be used interchangeably. It is understood that the term “bead” can connote, in many instances, a structure that is generally spherical in overall dimension. Beads, so configured, are contemplated by a variety of embodiments herein. As well, the present teachings contemplate a variety of embodiments employing beads (and the like) of various other shapes. For example, without limitation, beads for use herein include entities or constructs that are semi-spherical, oval, oblong, globular, granular, flake-like, pellet-like, etc. Beads, particles and the like, according to various embodiments, can comprise, e.g., organic and/or inorganic materials. Beads, particles, and the like, contemplated for use herein, can comprise entities or constructs which are solid (in whole or in part), substantially solid, and/or semi-solid. In some instances, beads, particles, and the like, may be gelatinous or fluid, at least in part. Various embodiments of the present teachings contemplate beads, particles, and the like, such as those comprised of glass, quartz, polymers, or a combination thereof. A variety of embodiments herein contemplate beads, particles, and the like, comprised of metallic materials (such as gold particles) and/or aromatic polymers (such as polystyrene beads). In some embodiments, beads, particles, and the like, comprise one or more of the following (instead of, or in addition to, the materials just mentioned): liposomes, polymers, nanocrystals such as quantum dots, and oil droplets. Quantum dots, according to certain embodiments, can be described, e.g., in U.S. Pat. Nos. 5,990,479 and 6,207,392 B1, and in “Quantum-dot-tagged microbeads for multiplexed optical coding of biomolecules,” Han et al., Nature Biotechnology, 19:631-635 (2001), incorporated herein by reference.

A variety of embodiments herein contemplate use of a plurality of supports (simultaneously and/or sequentially). It will be appreciated by those skilled in the art that beads, particles, or the like, of a population, may or may not be uniform or identical in all respects. For example, in a given population intended to comprise smooth, spherical beads, there may be differences (of varying degree) in one or more aspects of the beads, e.g., diameter, surface feature(s) (e.g., smoothness), etc. In these regards, it is noted that herein, various embodiments contemplate strict tolerances for beads, or the like, of a population, wherein the distribution among and between individual members of the population is narrow. Some embodiments, contemplate a narrow distribution among plural populations themselves (e.g., embodiments wherein batches of nucleic-acid bearing beads are analyzed in parallel and/or series). In various additional embodiments, wherein tolerances are not as strict, the distribution among and between individual support elements can be wider, though still relatively narrow. In other embodiments, a wide distribution may be acceptable.

It is noted that the size of a support element (e.g., bead or particle), as contemplated herein, can vary. For example, a support element, such as a bead, particle, and the like, can comprise a generally spherical shape and have a diameter (or mean diameter for one or more populations of beads), or other greatest dimension with regard to non-spherical support elements, within a range of from about 10 nm to about 100 nm, from about 100 nm to about 1000 nm, from about 1 μm to about 10 μm, from about 10 μm to about 100 μm, and up to 1 mm, or greater. In some embodiments, the diameter of the beads is within a range of from about 10 nm to about 100 nm, 100 nm to 1 μm, 1 μm to 10 μm, up to about 100 μm. In certain embodiments, the particles employed comprise a diameter within a range of from about 100 nm to about 10 μm.

The surface of beads or particles can be functionalized, according to various embodiments. Such functionalization of the beads can vary. For example, in a variety of embodiments, amine, carboxylic acid, biotin, and streptavidin can be functionalized on the particles for attaching capture probes.

It should be noted that the charges of a support element itself, such as a bead or particle, can vary. For example (in various embodiments), the charges are no greater than about 100, no greater than about 10,000, and/or no greater than about 1,000,000. In some embodiments, the charges can be greater than 1,000,000. The net charge of a particle bearing one or more capture probes can vary, as well. For example, there can be a net positive charge, no charge, or a net negative charge on the particles. In a variety of embodiments, the net charge is negative; e.g., in various embodiments, less than about 1 million negative charges.

A variety of embodiments, according to the present teachings, provide pattern arrays of tethered beads on a substrate for opposed-forces binding assays and detection, as taught herein. A number of such embodiments further provide spatially encoded tethered beads for assay multiplexing. In these regards, it is noted that with the many advances in solid-phase synthetic chemistry over the years, which has been employed to create certain microarrays for nucleic acid analysis, a number of opportunities are recognized herein to further realize benefits from such advances in chemistry. For example, various embodiments of the present teachings utilize such advances to create patterned arrays of tethered beads for opposed-forces assays and detection, as taught herein. For example, certain embodiments employ photocleavable amine-protecting o-nitrobenzyl groups that cleave on exposure to light at 365 nm as a starting material to be deposited on a solid substrate such as glass. Upon exposure through a photomask of a predefined pattern of spots, the illuminated regions (e.g., within a range of from about 1 μm to about 10 μm in diameter, with a spacing within a range of from about 20 μm to about 100 μm) are activated as the protecting groups are cleaved thus exposing the reactive amine groups for covalent coupling. The amine groups on the activated spots can be reacted to yield streptavidin capture sites on the surface. Upon introducing one type of tethered beads with the free end of each respective tether ending in a biotin linker, the tethered beads can be captured via high affinity binding of streptavidin to biotin. Because the active spots on the solid support can be made to approximately on the same order as the bead diameter, capturing of one bead decreases the probability of another bead being captured in the same spot due, for example, to steric hindrance. Once most or all of the spots have a captured bead, the excess beads can be removed from the chamber, and the remaining active sites can be rendered non-reactive by binding with a neutral biotin-linked moiety, such as polyethylene glycol (PEG). PEG, in various embodiments herein, also serves as a non-sticky coating to reduce non-specific binding of target nucleic acids to the solid support surface.

Further, a variety of embodiments contemplate multiplexing assays with spatial encoding. For example, a second photomask can be used to define a second set of patterned spots adjacent to the first set on the same solid support surface for capturing a second set of beads. This process can be repeated for additional sets of beads to achieve a very high levels of multiplexing, including beads for positive and negative controls as well as for assay calibration. In various embodiments, the entire bead array can be made very compact, with, e.g., about 10,000 beads occupying a footprint less than about 1 cm×1 cm with spacing between beads up to about 100 μm. Upon completion of fabricating the final sets of beads, remaining amine-protected sites on the glass surface can be activated and reacted with carboxylated PEG.

Some embodiments employ an array fabrication method for spatial encoding on glass comprising microcontact printing of specific DNA capture sequences, which in turn capture double stranded DNA (dsDNA) tethers with complementary single stranded DNA (ssDNA) dangling ends. Soft lithographic techniques, as known in the art, can be employed to provide features down to tens of nanometers, therefore is suitable for making compact arrays in a flow cell, as contemplated by various embodiments herein.

Those skilled in the art will appreciate that certain structural and chemical aspects of the just-described embodiments can be varied, without undue experimentation, to achieve various desired results. For example, modifications and variations can be made by those skilled in the art with regard to the amine-protecting o-nitrobenzyl groups, the specified spacing of capture sites, and the specified array size. These and other such modifications and variations are included within the scope of the present disclosure.

As indicated above, those skilled in the art can select various suitable supports, being guided by various characteristics, purposes, functions, and such, relating to support elements, as taught herein. For example, criteria in selecting a suitable support can include, among other things, the size and/or binding capacity for probes associated with the support; the nature or purpose of the support in such association (e.g., a means for immobilizing, or otherwise maintaining within a desired site, location or orientation, one or more probes); the charge(s),if any, of the support itself (particularly when exposed to conditions effective to change a property or characteristic upon a binding event).

Some embodiments contemplate the use of gold particles, beads, or the like. The gold beads can, for example, be characterized by low surface charge, a single polymer tether, and, optionally, one or more capture oligos (note, the term “oligo” is sometimes used as an abbreviation for “oligonucleotide”). In various embodiments, individual particles, of a plurality of particles used in a binding assay in accordance with the present teachings, includes 1 tether, and less than about 1,000 charges per bead.

Additional factors, which can be useful in the selection of supports, can include, for example, properties important to the interaction of the support with the selected forces and/or with the detection approach employed, such as density, dielectric constant, scattering cross-section, fluorescence, phosphorescence, polarizability, magnetic susceptibility, and/or electrical conductance. Furthermore, other considerations such as ease in surface modification, stiffness, surface energy, and barcoding capability (for example, to encode the identity of the source of the nucleic-acid molecules on the support) can also be relevant.

Various embodiments contemplate the use of plural particles. For example, at least about 10, at least about 50, at least about 100, at least about 1,000, at least about 10,000, at least about 100,000, at least about 1,000,000, and/or at least about 10,000,000 particles. For such embodiments utilizing plural particles, the particles can be disposed in any suitable arrangement, whereby opposing forces can act upon them and changes in a property of the beads can be detected.

In a variety of embodiments, a plurality of particles is configured so as to define an array. The quantity of particles included in an array can vary. In various embodiments, for example, an array of particles includes at least 10, at least 50, at least 100, at least 1,000, at least 10,000, at least 100,000, at least 1,000,000, and/or at least 10,000,000 particles. The configuration of an array can vary, too. In various embodiments, arrays can be, for example, substantially planar. Various embodiments contemplate generally two-dimensional arrays. In some embodiments a plurality of beads are disposed in a linear array. In various embodiments, for example, a two dimensional array of beads is used such that the beads are in a repeating pattern with a pitch of between 0.1 and 20 microns. In other embodiments, for example, the beads can be in an array where the beads are in a non-repeating pattern.

With regard to encoding, various embodiments contemplate a variety of encoding means. Suitable encoding means can be selected by those skilled in the art. Encoding means can include, for example and without limitation, encoded or labeled polymeric, ceramic, semiconductor, and metallic particles or beads, barcodes, barcoding schemes, encodable tags, encodable labels, molecular encoding, oligo- and polynucleotide-based encoding, etc. Codeable tags, or other encoding means, can be selected to be suitably “detectably different.” In other words, they can be selected so as to be distinguishable from one another by at least one detection method. In various embodiments, detection of a given codeable tag, for example, can indicate the presence of a respective moiety to which the codeable tag is specific; while the absence of a given codeable tag can indicate the absence of the moiety to which the codeable tag is specific.

Information encoded, for example, can be specific to a particular moiety (or, in some instances, more than one moiety). In various embodiments, the identity of the source of a nucleic-acid molecule on a support element can be encoded. In some embodiments, this can be done, for example, for a plurality of different molecules of interest, such as from unique sources or samples.

In various embodiments, encoding means are employed with a plurality of binding moiety (i.e. a molecule with specific affinity for the analyte of interest) carrying beads. In some embodiments, such plurality of beads is arranged so as to define an array (e.g., a planar array). According to a variety of embodiments, highly multiplexed analyses can carried out, substantially simultaneously (that is, in parallel). As well, detection of results can be carried out in parallel (e.g., employing an imaging apparatus). In various embodiments, micrometer- and nanometer-dimensioned encoded particles, beads, or the like, capable of, or adapted for, carrying biological molecules can be miniaturized and employed for multiplexing in an array-based format. In some embodiments, employing uniquely encoded particles tagged with specific recognition probes, a small amount of sample can be analyzed simultaneously for a plurality of targets.

Further regarding encoding, binding moiety support elements (e.g., particles carrying one or more binding moieties capable of recognizing one or more analytes of interest), according to a variety of embodiments, can be coded via position or placement. According to various embodiments, a plurality of nucleic-acid carrying particles is arranged in an addressable array (that is, an array having a known carrier particle associated with a known location (address) in the array).

Various approaches of encoding/decoding are described, for example, in A W Czarnik, “Encoding strategies in combinatorial chemistry,” Proceedings of the National Academy of Sciences of the United States of America, 94, no. 24 (1997): 12738-9; Braeckmans, K, and S C De Smedt. “Colour-coded microcarriers: Made to move.” Nature materials 9, no. 9 (2010): 697-8; Braeckmans, Kevin, Stefaan C. De Smedt, Marc Leblans, Rudi Pauwels, and Joseph Demeester. “Encoding microcarriers: present and future technologies.” Nat Rev Drug Discov 1, no. 6 (June 2002): 447-456; and, US Patent Publication Number 2005/0272049 A1, entitled, “Arrays of magnetic particles”; each incorporated herein by reference.

According to various embodiments, any suitable means can be employed to associate one or more selected probes with one or more particles for analysis in accordance with the present teachings. Probes can associate with particles in any suitable manner. For example, in various embodiments, such association can be by means of physical adsorption, covalent bonds or non-covalent binding.

With further regard to means for associating one or more binding moieties with one or more particles, according to various embodiments, a target binding moiety can be carried, attached, bound, fixed, linked, or otherwise maintained proximate to a selected particle

Some embodiments herein contemplate a direct association between a binding moiety (i.e. capture probe) and a particle. Some embodiments herein contemplate an indirect association of a binding moiety with a particle. In various embodiments, one or more appropriate linker moieties are employed as a means for associating a binding moiety and a particle. A variety of linkers can be utilized. For example, linkers, as contemplated by various embodiments here, can be stable or can be labile, as desired. In some embodiments, a cleavable linker associates a binding moiety with a particle. For example, among other suitable types, photocleavable linkers (PC-linkers) can be employed. Some embodiments contemplate use of a photocleavable biotin (PC-biotin) reagent as a means for releasably associating a binding moiety with a particle. For example, in some embodiments, a PC-biotin reagent can be conjugated to an appropriate material or species comprising a particle, such as a primary amine using standard and facile NHS chemistries. Subsequently, near-UV light can be used to break the link between the biotin and the particle, as desired.

In some embodiments, one or more suitable intervening or bridging structures and/or layers are employed between a nucleic-acid molecule and an associated particle. For example, in some circumstances, it can be desirable to have a binding moiety held or maintained in spaced-apart relation with respect to an associated particle. The spaced-apart relation can be fixed, substantially fixed, or it can be variable, as desired. In various embodiments, one or more suitable modifications are made to a particle (e.g., derivatization of a surface structure) for purposes of facilitating attachment. In other embodiments, the bridging structure between a nucleic-acid molecule and an associated particle can contain information of the identification (i.e., a barcode) of the origin of the nucleic-acid molecule and/or of the particle. The barcode can be a unique nucleic acid sequence, for example.

It should be noted that, herein, a distinction is generally not made regarding the type of association, direct or indirect, nor whether a binding moiety comprising a probe is in physical contact with a support element, such as a particle, bead, or the like, or maintained in spaced-apart relation. An exception being only where made clear that a distinction is pertinent and so deliberately intended. Thus, in using terms (or forms thereof) such as “associated,” “bound,” “carried,” “attached,” “linked” or the like, with regard to one or more binding moieties (e.g., oligonucleotide probes, antibodies, etc.) and one or more support elements (e.g., beads or particles); only unless otherwise made clear, details as to the nature of the association, such as being direct, indirect, touching, spaced-apart, or the like, are not intended and such terms are not to be understood as being limiting in these regards.

At this point, aspects of detection will be described. In accordance with the present teachings, a detector, detection assembly, and/or detection system or sub-system can be employed to detect, for example, one or more signals relating to one or more kinematic properties pertaining to one or more beads, particles, or the like. Properties can be measured by a variety of means including, for example, magnetic, electrical (e.g. Coulter counter), and/or optical detection. For example, according to various embodiments, an optical detection system can be employed to detect a signal relating to a property of a particle; and in some embodiments, a property for each of a plurality of particles (e.g., an array of particles). Detected properties can include, for example, position, velocity (linear or rotational), or acceleration. [See, e.g., “Particle Kinematics,” by E. Byckling and K. Kajantie, Published 1973 by Wiley in London; incorporated herein by reference].

Detectors, detection systems, and the like, according to various embodiments, can include or be operably linked to any suitable computer system, or other suitable logic device, e.g., via an analog to digital or digital to analog converter, for transmitting detected light data to the computer for performing various tasks, such as collection, analysis, manipulation, and storage of data.

Any suitable means for detecting signals (e.g., optical signals) can be utilized. For example, in various embodiments, the means for optical detection includes systems adapted for measuring transmitted, absorbed, scattered, polarized, phase-shifted and/or emitted light. In various embodiments, for example, microscopes for brightfield, phase contrast, DIC or fluorescence microscopy can be used. Various embodiments of the present teachings employ a means for optical detection configured to measure one or more particle parameters from an array of particles; such as, for example, arrays comprising more than 1, more than about 100, more than about 1,000, more than about 10,000, more than about 100,000, and/or more than about 1,000,000 particles. Suitable detectors can include, for example, light detectors, such as quadrant photodiodes, and in some embodiments, imaging systems, such as charged coupled devices (CCDs or other array detectors), and the like. In some embodiments, it is contemplated that non-optical means for detecting changes in particle property can be used to infer changes relating to binding events, such as by magnetic, electrical resistance, capacitance, impedance, and acoustics approaches.

In various embodiments, electrical methods and apparatus can be employed for detecting changes in the electrophoretic mobility of beads upon target capture of probes attached to the surface of the beads. As compared to certain optical detection technologies, this embodiment can provide for a more compact and inexpensive instrumentation for many applications, including point-of-care diagnostics. Further in this regard, it is noted that when beads with surface-bound probes capture target nucleic acids or proteins, for example, the changes in charge brought on by the target can change the Zeta potential of the beads, giving rise to a change in electrophoretic mobility. Such a change can readily be detected by optical means, such as by optical imaging, light scattering, fluorescence microscopy, or laser Doppler shift, with a high degree of accuracy. However, many of the optical approaches can be expensive and, in some instances, bulky components may be required to implement the detection.

For some applications contemplated in connection with the present teachings, such as point-of-care diagnostics, it can be desirable to employ very compact instrumentation as a reader. This can be particularly the case where it is desired to embody the present teachings in a hand-held device or apparatus. Certain embodiments herein employ methods that avoid the use of optical means for measuring microelectrophoresis, for example, by electrical resistance detection at two channel junctions through which a bead of interest crosses. In some embodiments, for example, as the bead moves along a main channel under electrophoretic motion, reaching a first junction and then a second junction, electrodes at the respective crossed channels record an increase in electrical resistance at two different times, the difference of which is the transit time of the bead. The junctions are configured with a separating distance between them that is known, so the measured transit time can be translated into bead velocity. Bead velocities before and after sample incubation, in embodiments such described here, can be compared to quantify the amount of target marker captured from the sample.

According to various additional embodiments, laser Doppler effects are used in detection, based on frequency or phase shift of scattering light, to measure changes in bead velocity during microelectrophoresis to assess whether a hybridization or binding event has occurred upon exposure of beads covered with capture probes to a sample of interest in opposed-forces sensing of nucleic acid, protein, small molecule markers, or other analyte of interest. In this regard, in Zeta potential measurements, one technique sometimes employed is a “Laser Doppler Frequency Shift” method where a part of a laser beam is split off and interferes with the beam interacting with the sample. Light scattered from a moving particle in the sample experiences a frequency shift. The instrument measures the frequency of the beating beam produced by the interferometric mixing of the reference and scattering beams, which determines the mobility of the particles. Another method sometimes used is “Phase Analysis Light Scattering (PALS)” which determines the phase difference of the sample interfering beam with the reference beam. The phase difference can be detected and quantified, for example, 100 times faster than the frequency shift, which means that mobility can be measured much more precisely using PALS than with the Doppler shift method. It is contemplated herein that one or both of these methods can be used in opposed-forces sensing, of the present teachings, to measure the onset, for example, of enzymatic or binding reactions on the surface of beads covered with specific capture probes. The incident beam frequency can be varied or altered and still accomplish the objective of good detection. Beads can be untethered or tethered with a long polymer chain that does not interfere with electrophoretic motions of the bead during the measurement period.

In a variety of embodiments, a detector assembly, such as an imaging array detector, can be configured in combination with a computer or other suitable logic device, with software for converting detector signal information into assay result information, or the like. For example, a computer system, such as a personal computer or Apple Macintosh (commercially available from Apple Inc.), can be programmed to convert detector signal information into assay result information providing information about the presence, absence, quantity, etc. of one or more analytes of interest. In some embodiments, a computer system is programmed to interpret detector signal information and provide output corresponding to the presence or absence of analytes for a selected panel of analytes of interest. Signal data and/or assay-result information can also be sent, as desired, to an output or storage device, such as a display device (monitor), a printer, and/or disk drive.

A variety of programming means can be utilized, in accordance with various embodiments of the present teachings. In a variety of embodiments, e.g., sets of instructions for selected process steps can be written, for example, using C programming language (e.g., in some embodiments, a program is written in C for calculating from a CMOS sensor images of one or more analyte-bearing beads, positional information). Other programming means can be employed, as well. In various embodiments, a control computer can integrate the operation of various assemblies, for example through a program written in an event driven language such as LABVIEW® or LABWINDOWS® (National Instruments Corp., Austin, Tex.). In particular, the LABVIEW software provides a high level graphical programming environment for controlling instruments. U.S. Pat. Nos. 4,901,221; 4,914,568; 5,291,587; 5,301,301; 5,301,336; and 5,481,741 (each expressly incorporated herein by reference) disclose various aspects of the LABVIEW graphical programming and development system. The graphical programming environment disclosed in these patents allows a user to define programs or routines by block diagrams, or “virtual instruments.” As this is done, machine language instructions are automatically constructed which characterize an execution procedure corresponding to the displayed procedure. Interface cards for communicating the computer with the motor controllers are also available commercially, e.g., from National Instruments Corp.

Some embodiments employ a software tool, previously described, that facilitates and automates feedback control of an optical trap for dynamic single-molecule tethered-bead studies (See, e.g., Steven J. Koch (University of New Mexico, Department of Physics & Astronomy and Center for High Technology Materials), and Richard C. Yeh, (New York), “Versatile Control System for Automated Single-Molecule Optical Tweezers Investigations,” NY; Nature Preceedings : dl:10101/npre.2010.4284.1 : Posted 16 Mar. 2010; http://precedings.nature.com/documents/4284/version/1/files/npre20104284-1.pdf; incorporated herein by reference). The latter provides a versatile control system to automate single-molecule biophysics experiments. The method combines low-level controls into various functional, user-configurable modules, which can be scripted in a domain-specific instruction language. The ease with which the high-level parameters can be changed accelerates the development of a durable experiment for single-molecule samples. Once the experimental parameters are tuned, the control system can be used to repeatedly manipulate other single molecules in the same way, to accumulate the statistics to report results from single-molecule studies. (National Instruments LabVIEW 7.1 and D, and LabVIEW 6.1. versions, for example, are available from SourceForge, and are described on an OpenWetWare site: https://sourceforge.net/projects/tweezerscontrol/).

The methods, systems and apparatus of the present teachings, in a variety of embodiments, provide binding assays wherein detection is carried out while opposed forces act upon capture complexes. A low-cost trap can hold each complex. Nucleic acids (or other markers) of specific pathogens can be detected with good sensitivity and specificity provided, at least in part, by charge-label signal enhancement. The present teachings can be used, for example, to identify multiple pathogens, distinguish pathogen strains, recognize early infection, and detect drug sensitivity and resistance. The benefits of such a sensitive, inexpensive, and easy-to-use diagnostic system include substantially reducing or eliminating the potential for sample cross-contamination due to target amplification, and minimizing the need for highly trained personnel. The methods, systems and apparatus of the present teachings can significantly improve the timeframe for “sample-to-answer,” and can provide nicely reliable results in near-patient settings. Embodiments having some or all of these benefits can be expected to drive commercial adoption in molecular pathology laboratories in clinics and small to medium sized hospitals. The resulting increased timeliness and accuracy of treatment decisions can be expected to improve patient outcomes and save overall healthcare costs.

An exemplary application of the methods, systems and apparatus herein are contemplated by certain embodiments that provide for the use of opposed-forces binding assays for sensing to detect the presence of urease. The detection of urease can be used as a diagnostic to detect presence of pathogens. Urease is an enzyme that catalyzes the hydrolysis of urea into carbon dioxide and ammonia. Rapid urease test, also known as the CLO test (Campylobacter-like organism test), is a rapid test for diagnosis of Helicobacter pylori. In this test, a biopsy of mucosa is taken from the antrum of the stomach, and is placed into a medium containing urea and an indicator such as phenol red. The urease produced by H. pylori hydrolyzes urea to ammonia, which raises the pH of the medium, and changes the color of the specimen. Similarly, in opposed-forces binding assays and detection, the increase in pH can change the Zeta potential of a bead, enabling a sensitive and rapid detection of the H. pylori. Urease-positive pathogens include, for example, Helicobacter pylori; Enteric bacteria including Proteus, Klebsiella and perhaps Serratia; Ureaplasma urealyticum, a relative of the mycoplasma; Cryptococcus, an opportunistic fungus; and Proteus mirabilis.

Additionally, it is contemplated that urease can also be used as the enzyme component of an ELISA assay for high sensitivity ELISA.

As discussed above, the known present-day diagnostic platforms and approaches tend to be either (a) generally straightforward to use and acceptably inexpensive (e.g., lateral flow immunoassays), yet suffer from poor sensitivity, or (b) quite sensitive, but very expensive and difficult to operate (e.g., nucleic acid amplification assays). In a variety of circumstances, such as in the near-patient setting, the need remains for a platform that is compact and inexpensive.

Those skilled in the relevant arts will appreciate, e.g., from the embodiments set out herein, and the examples that follow, that various aspects of the present teachings provide at least some, and in various embodiments all, of the attributes generally understood as being highly desirable, while not being burdened (especially to the extent as the systems to date) with regard to negative attributes, such as high complexity and cost. For example, the present teachings provide a variety of embodiments comprising diagnostic platforms and approaches that perform well in terms of sensitivity (i.e., acceptable for their intended purpose(s)) and, as well, are generally easy to use and inexpensive.

By way of the present teachings, for example, assays for one or more analytes of interest can be carried out without the use of extrinsic labels for detection (in other words, free of extrinsic labels, such as fluorophores). Thus, the cost of reagents can be greatly reduced compared to known, conventional systems that are dependent upon extrinsic optical labels. Further, various embodiments of the present teachings can employ simple and relatively inexpensive dark-field optics. Also, an ordered bead array, according to various embodiments, can facilitate the efficient use of pixels in an imaging detector. A system of the present teachings can cost, for example, substantially less than the prior or existing commercially available systems (e.g., those employing fluorescent labels and PCR) which, as discussed above, although providing good sensitivity, generally suffer from very high cost and complex use.

It will be appreciated that the present teachings provide, among other things, effective and efficient assays for one or more analytes of interest by monitoring changes in a parameter or property of a probe-bearing support element as the balance of forces acting thereupon changes due to specific binding events when an analyte-containing sample is introduced. Various aspects of opposed-forces binding assays, as embodied in various methods and systems herein, can be employed, for example, in diagnostic platforms useful with regard to infectious diseases such as those discussed above.

Studies and first-principle calculations indicate that opposed-forces binding assays can significantly reduce the cost of diagnosis and treatment, as compared to prior, known, and commonly employed approaches. Various aspects of the present teachings can avoid, or otherwise overcome, certain key issues and challenges faced by the platforms and approaches to date.

In this regard, various aspects of the present teachings provide for: (1) sample preparation that does not involve one or more amplification steps such as PCR; (2) label-free detection, which avoids various costly sequencing reagents used in various conventional systems; (3) relatively simple optics, comprised of readily commercially available components, thereby providing for a relatively low instrument cost; (4) highly parallel multiplexing (as by way of bead arrays), which provides for high throughput and, consequently, a low capital equipment amortization cost per assay; and (5) acceptable sensitivity for their intended purpose(s). Low-cost systems and methods realizing some or all of the just-mentioned benefits (e.g., various embodiments of opposed-forces binding-assay systems and methods, herein) will greatly benefit clinical care.

### EXAMPLES

The present teachings may be further understood in light of the following examples, which are illustrative and are not intended in any manner to limit the scope of the present teachings or claims directed thereto.

### Example 1

Low-Cost Point-of-Care Diagnostics for Multiplexed Analyte Detection

This example is illustrative of a sensitive, flexible, and cost-effective point-of-care (POC) diagnostic platform for the simultaneous detection of multiple analytes, including DNA, RNA and protein biomarkers, in urine and other fluids derived from a patient. The present example contemplates a generic, label-free detection approach employing opposing electrical, hydrodynamic, and restoring forces on a bead to measure changes in the surface charge of the bead due to analyte binding. Further, the present example contemplates an apparatus comprising a compact, battery-powered platform configured for multiplexed test panels for the analysis of diarrhea, HIV/AIDS, malaria, pneumonia, and tuberculosis from urine or other patient fluids. The method and apparatus of this example involve minimal sample processing for quantitative diagnostics, and facilitate adequately reliable and timely clinical decisions.

Briefly, by way of background, there are many examples of using non-invasive urine samples to detect global health conditions. For example, immunoglobulin A specific to Campylobacter which causes diarrhea has been detected by Western blotting [See S-J Wu, et al., J. Clin. Microbiol. 31:1394 (1993)]. HIV antibodies have been detected by the Seradyn immunoassay which has 99% sensitivity and 98% specificity [See A. Carducci, et al., Eur. J. Epidemiology, 15:545 (1999)], Streptococcus Pneumoniae antigen associated with pneumonia has been detected using Binax's immunochromatographic test with good clinical validity [D R Boulware, et al., J Infect. 55:300 (2007)], while mycobacterium tuberculosis and malaria DNA were detected by PCR [See A. Aceti, et al., Thorax 54:145 (1999); and see S. Mharakurwa, et al., Malar J. 5:103 (2006)]. While these urine-based tests look promising, many still lack sufficient sensitivity or require trained personnel and an expensive instrument. Moreover, the need to run different tests on different platforms often delays and increases the costs of accurate diagnosis of a patient with clinical symptoms shared by multiple causes. Therefore, a single, flexible platform enabling field-adapted diagnostic test panels for multiple diseases based on both nucleic acid and protein biomarker detection, as provided in this particular example of the present teachings, is desirable to enhance clinical sensitivity and specificity, as well as to reduce time and cost per clinical decision.

The illustrative system, apparatus, and method of this example provide for the sensitive detection of multiple analytes. As contemplated by this example, a particle moves in response to an electrical force which is proportional to the number of charged analytes bound to the particle, and is opposed by a hydrodynamic force and a restoring force. Nucleic acids or antibody capture probes are attached to the bead surface to capture nucleic acid or protein biomarkers leading to detection by the change in charge. Polymer tethers, which trap a complex comprising a bead having surface-bound probes, are employed to provide low-cost, multiplexed analysis. Specific capture of nucleic acids or proteins from the patient urine sample onto the particles changes the balance of the forces, leading to a measurable change in bead displacement. Simple dark-field optics can be employed for the measurement. The samples assayed can be crude urine samples. Such features provide for a relatively low-cost instrument having essentially fully self-contained functionalities (e.g., camera, power supply, computer, user interface, etc.) currently available in inexpensive mobile phones and a compact footprint facilitating portability for use, for example, in remote regions with limited or no access to electrical power.

The instrument platform of this example can adapt and expand its test menu readily as more urine-based clinical assays are developed and validated. Disease panels can be configured to suit the needs of a specific endemic locale by incorporating beads with specific capture probes in an inexpensive disposable cartridge. The instrument cost for this platform can be comparatively low because essentially all components, including the inexpensive CCD for detection, are available as off-the-shelf items that are presently manufactured in large volumes.

This illustrative example contemplates reagents, conditions, and the like, for label-free detection of nucleic acids or proteins on a single bead using a polymer tether instead of an optical trap as a restoring force. Further, this example contemplates a flow cell, such as embodied in a microfluidic card or device, capable of capturing analytes flowing past a bead array, with each bead of the array tethered to a respective analysis site on a surface feature (e.g., a floor or wall) of an analysis region in the flow cell.

To prepare beads, care is taken to keep the native charge of the beads at or close to neutral in order to enhance or maximize the sensitivity and/or dynamic range of the charge change measurements on the bead. For example, carboxylated polystyrene beads (e.g., having a mean diameter within a range of from about 1 μm to about 5 μm) can be functionalized via carbodiimide chemistry by reacting with groups containing amines. Capture probes and a polymer tether can be attached to each bead by mixing the polystyrene beads with: (1) a low stoichiometric ratio of a tether double stranded DNA (dsDNA) such that Poisson statistics favors one or zero tether per bead; (2) an amine-labeled primer hybridized to a template oligonucleotide or a monoclonal antibody; and (3) excess ethanolamine to neutralize remaining unreacted carboxylic acids on the beads. The dsDNA tether can be constructed using amine and biotin labeled nucleotides [See, for example, R M Zimmerman & E C Cox, Nucleic Acids Res. 22:492 (1994), and see M Usdin, et al., Nature, 44:283 (2006); each of which is incorporated herein by reference].

The present illustrative example, as well, provides for an apparatus using a quadrant photodiode to measure the bead displacement of a tethered bead (e.g., similar to the arrangement successfully used by Galneder et al. [See R. Galneder, et al., Biophys J. 80:2298 (2001), incorporated herein by reference]. Different voltage conditions are applied in the method and apparatus of this example (e.g., DC to stretch the tethered chain and AC to move the beads) to enhance or optimize the detection sensitivity. This example further contemplates a flow-through microfluidic bead chamber in which beads are anchored at respective analysis sites in a detection area or region of the chamber, and analyte solutions potentially including one or more specific DNA and/or proteins of interest that can come in contact with the bead array. To optimize for variations of the method, apparatus, and system described in this example, as well as for any of a wide variety of analytes to be assayed, concentrations of analytes and flow rates can be tested to determine acceptable or optimal capture conditions.

The method and apparatus of this example provide, among other things: (1) a simple and robust protocol for preparing tethered beads in a flow cell, (2) optimized conditions for capturing nucleic acid- or protein-based analytes from solutions onto the beads, and (3) optimized conditions for measuring changes in bead displacement with acceptable accuracy and precision upon binding events on bead surfaces.

The system of this example provides for inexpensive, flexible and, as desired, highly multiplexed assays. As well, it can be configured to use disposable cartridges and adapted for point-of-care (POC) use. In short, the system of this example is a significant advancement in the field of diagnostics because, for example, it is inexpensive, easy-to-use, portable, and robust. Further, the system employs optimized protocols for generating high-density tethered bead arrays for multiplexed analysis. Additionally, the system employs an inexpensive optical detection subsystem for monitoring multiple bead displacements in parallel and optimized conditions to detect, for example, at least two high priority global health diseases in urine with minimal sample preparation [See, for example, M. Usdin, et al., Nature, 44:283 (2006), incorporated herein by reference].

### Example 2

A Highly Specific and Sensitive Binding Assay Employing Opposed-Forces Sensing

This example describes illustrative set-ups and methods for determining the effectiveness of a signal-amplification scheme, consistent with the teachings herein, aimed at providing a substantial increase in specificity as well as sensitivity for binding assays. In the approach of this example, a particle moves in response to an electrical force which is proportional to the number of charged analytes bound to the particle, and is opposed by a hydrodynamic force and a restoring force. Nucleic acid capture probes are attached to the bead surface to capture pathogen nucleic acid fragments through base-pairing hybridization, leading to detection by the change in charge. The use of branched DNA (bDNA) probes amplifies the signal (not the target) by increasing the total charge on the bead, thereby increasing the sensitivity and specificity of the assay to approach, and perhaps match, those of PCR-based assays but without the need for target nucleic acid amplification.

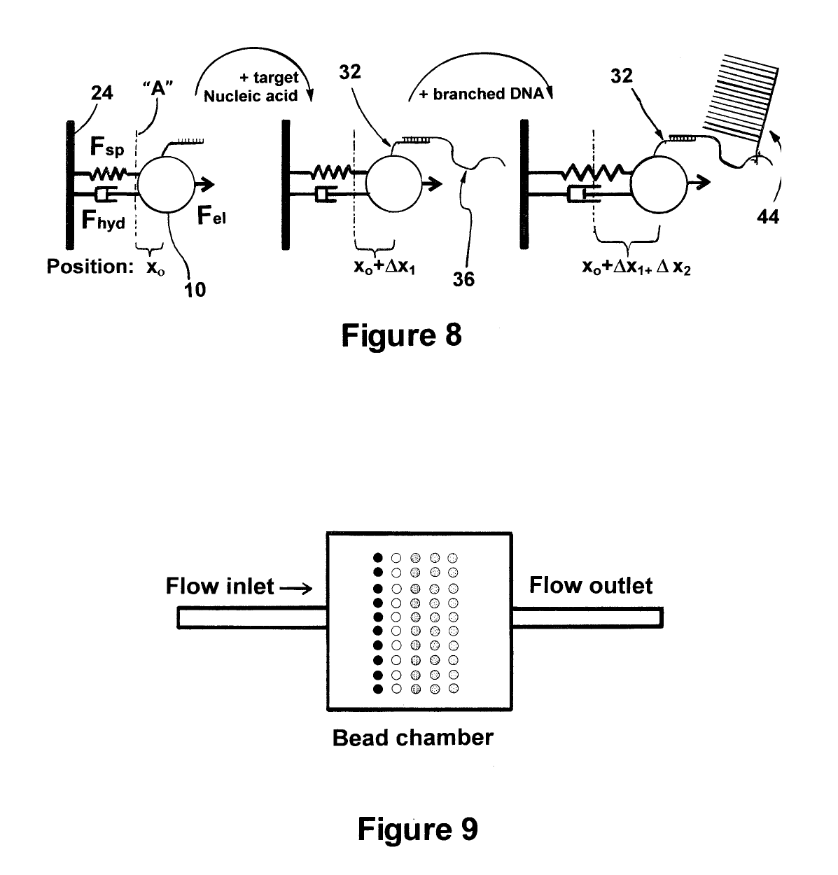

Next, this example provides an illustrative system and method for demonstrating a binding assay of the present teachings, as well as a description of expected results and mechanisms of action underlying the results. It is proposed in this example to monitor the capture of specific oligonucleotides onto bead-attached DNA by virtue of changes in the number of charges on a bead. Forces are used to act on the bead, which comprise: (1) an electrical force which is directly dependent on the total electrical charge of DNA on the bead, and (2) opposing forces consisting of a hydrodynamic force and a restoring spring-like force provided by a tethered polymer, which do not depend strongly on the electrical charge of DNA on the bead surface. These applied force vectors are shown schematically in FIG. 2. A more detailed depiction of the proposed set-up, shown in FIG. 7, illustrates a bead covered with DNA capture probes and attached to a surface via a dsDNA tether. In this detection approach, hybridized oligonucleotides from a patient sample can be expected to change the balance of the forces, leading to a measurable change in bead position or velocity in an electric field. In addition, this example contemplates the use of branched DNA hybridized to the target nucleic acid captured on beads to enhance the change in charge and thus detection sensitivity and specificity to approach or match the performance of PCR assays (see FIG. 8). The use of polymer tethers is expected to provide for the cost-effective fabrication of large bead arrays for development of a multiplexed testing panel.

Simple dark-field optics are contemplated for measurement, which a low-cost instrument which we envision would have fully self-contained functionalities (camera, power supply, computer, user interface, etc.) currently available in inexpensive mobile phones, and a compact footprint to be truly portable for point-of-care use. The instrument cost for this platform is expected to be very low because all components, including the inexpensive CCD for detection, are available as off-the-shelf items that are already manufactured in large volumes.

Based on first-principle calculations which are described in the following example, it is believed that the present opposing-forces detection method may closely approach, or possibly reach, the goal of an ideal point-of-care diagnostics because this technology is expected to perform well with regard to at least some, and possibly all, of the following attributes: low copy number or single molecule analysis, fast time-to-answer, high degree of multiplexing, no cross-contamination from amplified target DNA, and low cost instrumentation.

### Example 3

A Fluidic Device for Binding Assay Employing Opposed-Forces

Assays as described herein can be carried out, for example, in a microfluidic device or card, such as shown schematically in FIG. 9. This example contemplates a simple bead chamber comprising an inlet channel and an outlet channel, each adapted for fluidic communication with a central bead chamber, to provide for introduction of reagents and beads quickly and easily via an external pump, such as a Watson-Marlow syringe pump. FIG. 9 further shows, in schematic form, a patterned bead array that is fabricated inside a flow cell. The chamber is mounted on the Nikon Diaphot 300 microscope for evaluation of the spatial encoding results after each successive set of beads is tethered onto the glass surface.

While the principles of the present teachings have been illustrated in relation to various exemplary embodiments shown and described herein, the principles of the present teachings are not limited thereto and include any modifications, alternatives, variations and/or equivalents thereof. All such modifications, alternatives, and equivalents are intended to be encompassed herein.

This application incorporates by reference in their entirety for all purposes all publications, patents, and patent applications cited herein.

It is claimed:

1. A method to assay at least one biological analyte of interest in a fluidic sample, comprising:
(i) preparing the sample for analysis;
(ii) generating a complex comprising a particle, an associated biomolecular recognition element capable of selective binding with the analyte, and a number of associated charges; wherein the complex is characterized by at least one kinematic property that depends on the number of associated charges;
(iii) anchoring the complex to a surface of an analysis region with a polymeric tether;
(iv) acting upon the tethered complex with opposing forces comprised of a plurality of forces that include vector components disposed in opposing directions; wherein at least one of the forces depends on the number of associated charges and at least another of the forces comprises a spring-like restoring force; and wherein the polymeric tether provides, at least in part, the restoring force;
(v) detecting for signals at the analysis region corresponding to the kinematic property, to obtain a first measurement;
(vi) contacting the complex with the prepared sample under conditions favorable for selective binding between the molecular recognition element and the analyte; wherein, in the event of such binding, the number of associated charges is changed;
(vii) acting upon the tethered complex with opposing forces comprised of a plurality of forces that include vector components disposed in opposing directions;
(viii) detecting for signals at the analysis region corresponding to the kinematic property, to obtain a second measurement; and
(ix) comparing the first and second measurements to determine a difference; whereby a non-zero difference is indicative of the presence of the analyte.

2. The method of claim 1, wherein said biomolecular recognition element comprises at least one capture probe supported by said particle.

3. The method of claim 2, wherein said at least one capture probe comprises at least one oligonucleotide.

4. The method of claim 1, wherein said polymeric tether comprises a biological polymer.

5. The method of claim 4, wherein said biological polymer comprises a protein or DNA.

6. The method of claim 1, wherein said kinematic property is position, linear velocity, rotational velocity, acceleration, or a combination of the foregoing.

7. A method to assay a biological analyte of interest in a fluidic sample, comprising:
(i) preparing the sample for analysis;
(ii) generating a complex comprising a particle, an associated biomolecular recognition element capable of selective binding with the analyte, and a number of associated charges; wherein the complex includes at least one kinematic property that depends on the number of associated charges;
(iii) trapping the complex at an analysis region;
(iv) contacting the complex with the prepared sample under conditions favorable for selective binding between the molecular recognition element and the analyte; wherein, in the event of such binding, at least one complex-bound analyte is formed, thereby changing the number of associated charges and forming a first charge-modified complex;
(v) acting upon the trapped complex with opposing forces comprised of a plurality of forces that include vector components disposed in opposing directions; wherein at least one of the forces depends on the number of associated charges and at least another of the forces comprises a spring-like restoring force provided by the trap;
(vi) detecting for signals at the analysis region corresponding to the kinematic property, to obtain a first measurement;
(vii) enhancing changes to the number of associated charges from step (v), if any, by providing in the sample a charged binding agent having a specific affinity for the complex-bound analyte, under conditions favorable for binding to thereby form a second charge-modified complex;
(viii) acting upon the trapped complex with opposing forces comprised of a plurality of forces that include vector components disposed in opposing directions;
(ix) detecting for signals at the analysis region corresponding to the kinematic property, to obtain a second measurement; and
(x) comparing the measurements to determine a difference; whereby the event of a non-zero difference is indicative of the presence of the analyte.

8. The method of claim 7, wherein the trapping is effected at least in part by a restoring force; wherein said restoring force is a tethering force, an optical trapping force, a magnetic trapping force, a dielectrophoretic trapping force, or a combination thereof.

9. The method of claim 7, wherein said biomolecular recognition element comprises at least one capture probe supported by said particle.

10. The method of claim 9, wherein said at least one capture probe is a nucleic acid, an antibody, an antigen, a protein, or any combination thereof.

11. The method of claim 10, wherein said at least one capture probe is comprised of at least a first nucleic-acid polymer and said analyte is comprised of at least a second nucleic-acid polymer, with said first and second nucleic-acid polymers including substantially complementary regions adapted, under suitable conditions, to hybridize to one another.

12. The method of claim 11, wherein said charged binding agent is comprised of at least a third nucleic-acid polymer.

13. The method of claim 12, wherein said binding steps of the method comprise hybridization; and wherein the charged binding agent comprises branched DNA (bDNA); and further wherein, after the analyte is bound to the capture probe, the branched DNA is bound to the analyte by specific hybridization in at least one region which is not occupied or hindered by the capture probe.

14. A method to assay at least one biological analyte of interest in a fluidic sample, comprising:
(i) preparing the sample for analysis;
(ii) generating a plurality of complexes, with each complex comprising a particle, an associated biomolecular recognition element capable of selective binding with the analyte, and a number of associated charges; wherein each complex includes at least one kinematic property that depends on the number of associated charges;
(iii) trapping the complexes at respective analysis sites collectively defining an array;
(iv) acting upon the plurality of trapped complexes with opposing forces comprised of a plurality of forces that include vector components disposed in opposing directions; wherein at least one of the forces depends on the number of associated charges and at least another of the forces comprises a spring-like restoring force provided by the trap;
(v) imaging the array of analysis sites to detect for signals corresponding to the kinematic property, to obtain a first measurement for each complex;
(vi) contacting the plurality of complexes with the prepared sample under conditions favorable for selective binding between the molecular recognition elements and the analytes; wherein, in the event of such binding for any complex, at least one complex-bound analyte is formed, thereby changing the number of associated charges and forming a first charge-modified complex;
(vii) acting upon the trapped complexes with opposing forces comprised of a plurality of forces that include vector components disposed in opposing directions;
(viii) imaging the array of analysis sites to detect for signals corresponding to the kinematic property, to obtain a second measurement for each of the complexes; and
(ix) comparing the first and second measurements to determine a difference for each of the complexes; whereby the event of a non-zero difference is indicative of the presence of the analyte.

15. The method of claim 14, wherein the method comprises a multiplexed assay, with said methods steps carried out on said plurality of complexes in parallel; and wherein said plurality of complexes includes at least 10, at least 100, at least 1,000, or at least 10,000 complexes.

16. The method of claim 15, wherein a number of complexes of the plurality of complexes are uniquely encoded, so that the complexes can be mixed and subjected to the assay of said method simultaneously, and subsequently identified by decoding.

17. The method of claim 14, wherein one or more of the traps comprises a restoring force; wherein said restoring force is a tethering force, an optical trapping force, a magnetic trapping force, a dielectrophoretic trapping force, or a combination thereof.

18. A method to assay an analyte of interest in a fluidic sample, comprising:
(i) preparing the sample for analysis;
(ii) generating a complex including a molecular recognition element capable of selective binding with the analyte;
(iii) contacting the complex with the prepared sample under conditions favorable for selective binding between the molecular recognition element and the analyte; whereby, in the event of such binding, a complex-bound analyte is formed;
(iv) providing a binding agent including a cleavable detection agent having a specific affinity for the complex-bound analyte, under conditions favorable for binding to the analyte;
(v) detecting for signals corresponding to a property of the cleavable detection agent, to obtain a first measurement;
(vi) cleaving and washing away the specifically bound cleavable detection agent;
(vii) detecting for signals corresponding to a property of the cleavable detection agent, to obtain a second measurement; and
(viii) comparing the first and second measurements to determine a difference; whereby the event of a non-zero difference is indicative of the presence of the analyte.
